(19) United States
(12) Patent Application Publication
(10) Pub. No.: US 2022/0265157 A1
(43) Pub. Date: Aug. 25, 2022

(54) SYSTEMS, DEVICES, AND METHODS FOR WIRELESS MONITORING

(71) Applicant: uLink Labs, Inc., San Mateo, CA (US)

(72) Inventors: Jayant CHARTHAD, Menlo Park, CA (US); Marcus WEBER, San Francisco, CA (US)

(21) Appl. No.: 17/496,216

(22) Filed: Oct. 7, 2021

Related U.S. Application Data
(63) Continuation of application No. PCT/US2020/027468, filed on Apr. 9, 2020.
(60) Provisional application No. 62/869,813, filed on Jul. 2, 2019.
(60) Provisional application No. 62/832,889, filed on Apr. 12, 2019.

(51) Int. Cl. A61B 5/0285; A61B 5/00; G16H 40/67; G16H 50/30

(52) U.S. Cl. CPC A61B 5/0285; A61B 5/0004; A61B 5/0022; G16H 40/67; G16H 50/30; A61B 2562/0204

(57) ABSTRACT

Described here are wireless monitoring devices, systems, and methods for estimating one or more physiological parameters of a patient. These devices and systems may measure or receive a signal waveform transmitted through one or more of fluid and a physiological structure of a patient. This measured signal waveform may be processed to generate waveform parameter data used to estimate a physiological parameter such as blood velocity, heart wall thickness, and the like.

### CROSS-REFERENCE TO RELATED APPLICATIONS

This application is a continuation of International Patent Application No. PCT/US2020/027468, filed Apr. 9, 2020, which claims priority to U.S. Provisional Application No. 62/832,889, filed Apr. 12, 2019, and U.S. Provisional Application No. 62/869,813, filed Jul. 2, 2019, each of which is hereby incorporated by reference in its entirety.

### TECHNICAL FIELD

Devices, systems, and methods herein relate to wireless monitoring to estimate one or more physiological characteristics and parameters of a patient.

### BACKGROUND

Monitoring of physiological parameters of a patient, such as blood velocity, a parameter related to a cardiac structure or an implantable device, and the like, may be useful for the diagnosis and/or monitoring of diseases such as heart failure, prosthetic valve dysfunction, valvular heart disease, restenosis, and the like. For example, monitoring of parameters such as blood pressure in the left ventricle (LV) and/or left atrium (LA), in the right ventricle (RV) and/or right atrium (RA), blood pressure and/or velocity in the pulmonary artery (PA), characteristics of a heart wall (e.g., thickness, motion), blood velocity or flow through one or more cardiac chambers or structures (e.g., left ventricular outflow tract), and the like, may be used for the diagnosis and/or monitoring of heart failure and/or other cardiovascular (CV) diseases.

Monitoring of a physiological parameter of a patient is typically performed using imaging techniques, an example of which is transthoracic echocardiography (TTE), and/or using catheters. However, such approaches may require resource-intensive procedures, may be time consuming, may require expertise to interpret imaging data, and results may depend on operator skill. Furthermore, data obtained from external imaging techniques may not offer sufficient resolution or accuracy needed for a reliable diagnosis or monitoring of a disease. As such, additional devices, systems, and methods for the estimation of a physiological parameter of a patient may be desirable.

### SUMMARY

Described here are wireless monitoring devices, systems, and methods for monitoring one or more physiological parameters of a patient. These devices and systems may, for example, receive a signal waveform transmitted through one or more of a fluid and a physiological structure of a patient. This signal waveform may be processed to generate a set of waveform features, or waveform parameter data, used to estimate a physiological parameter such as blood velocity, heart wall thickness, and the like. The devices described herein may have low energy requirements as well as a compact and implantable form that may allow for continuous or semi-continuous monitoring of the patient. For example, one or more wireless monitors may be disposed on an expandable cardiac implant such as a prosthetic valve or a stent, and may be used to monitor one or more of blood velocity, blood pressure, operation of the prosthetic valve or stent, and the like, over time.

In some variations, a wireless monitoring system is provided, comprising a wireless monitor including a first transducer configured to measure a signal waveform transmitted through one or more of fluid and a physiological structure of a patient. The wireless monitor may further include a first processor configured to process the measured signal waveform to generate waveform parameter data. The wireless monitoring system may further comprise a wireless device including a second processor configured to estimate a physiological parameter of a patient based on the waveform parameter data.

In some variations, a wireless monitoring system is provided, comprising a wireless monitor including a first transducer configured to measure a signal waveform transmitted through one or more of fluid and a physiological structure of a patient. The wireless monitor may further include a processor configured to process the measured signal waveform to generate waveform parameter data, and to estimate a physiological parameter of the patient based on the waveform parameter data.

In some variations, the fluid may comprise blood and the physiological structure may comprise one or more of a cardiac structure, a vascular structure, and a structure of a cardiovascular implantable device.

In some variations, the waveform parameter data may comprise one or more of a Doppler shift, a frequency shift, a phase shift, and a time delay, and the physiological parameter may comprise a fluid velocity. In some variations, the wireless monitor may comprise a second transducer positioned approximately opposite to the first transducer on or near a vessel wall. The second transducer may be configured to transmit a signal waveform and the first transducer may be configured to receive a reflected signal waveform, reflected at least in part by fluid flowing through the vessel. In some variations, the first and second transducers may be ultrasonic transducers. In some variations, a signal roundtrip time may be used to set the position of one or more reflection locations in the vessel, as described herein. In some variations, the first and second transducers may be configured to perform one-way or two-way pitch catch measurements for off-angle Doppler estimation of fluid velocity, as described in more detail herein. In some variations, the first and second transducers may be configured to perform pulse-echo measurements for off-angle Doppler estimation of fluid velocity. In some variations, the wireless monitoring system may comprise a second wireless monitor comprising a second transducer. The second transducer may be configured to transmit a signal waveform and the first transducer may be configured to receive a reflected signal waveform, reflected at least in part by fluid flowing through the vessel. In some variations, the wireless monitoring system may comprise a wireless monitor including one or more arrays of transducers to beamform or focus transmitted and/or received signal waveforms at one or more desired reflection locations. In some variations, the wireless monitor may comprise one or more transducers tilted or oriented to point the main lobe of the transducers' radiation pattern towards, or approximately towards, one or more reflection locations. In some variations, the wireless monitor may comprise one or more transducers tilted or oriented to point the main lobe of the transducers' radiation pattern approximately parallel to the length of the vessel.

In some variations, the signal waveform may comprise a set of pulses having a pulse repetition period, and the waveform parameter data may comprise a set of pulse arrival times of the signal waveform.

In some variations, the signal waveform may comprise a continuous wave signal with a carrier frequency, and the waveform parameter data may comprise a set of phase shifts of the signal waveform relative to one or more reference phases of the signal waveform.

In some variations, the waveform parameter data may comprise one or more of local density of blood and number of one or more types of cells or contents in blood, and the physiological parameter may comprise blood velocity.

In some variations, the waveform parameter data may comprise one or more transit times of the signal waveform, and the physiological parameter may comprise a fluid velocity.

In some variations, the signal waveform may be transmitted toward a cardiac structure, and the physiological parameter may comprise a cardiac structure parameter. In some variations, the signal waveform may comprise one or more reflected pulses, and the waveform parameter data may comprise one or more time durations corresponding to the one or more reflected pulses. In some variations, the cardiac structure may comprise one or more of an anatomical structure of the heart and material build up in the heart. In some variations, the cardiac structure parameter may comprise a heart wall thickness, and the heart wall thickness may be estimated based on the one or more time durations corresponding to the one or more reflected pulses. In some variations, a wireless monitor may have a part that may be positioned inside a heart wall for a measurement of pressure inside the heart wall. In some variations, the cardiac structure may comprise a heart chamber and the cardiac structure parameter may comprise a volume of the heart chamber. In some variations, the cardiac structure may comprise one or more valve leaflets, and the physiological parameter may comprise one or more of valve leaflet motion, thickness, and deterioration.

Also described here are methods. In some variations, a method of estimating fluid velocity may comprise receiving or measuring a signal waveform transmitted through patient fluid, measured by a wireless monitor. The measured signal waveform may be processed to generate waveform parameter data. The fluid velocity of the patient may be estimated based on the waveform parameter data. In some variations, the method may comprise estimating fluid velocity using a plurality of transducers with geometric symmetry for off-angle Doppler measurements, as described herein.

In some variations, a method of estimating a cardiac structure parameter may comprise receiving or measuring a signal waveform transmitted through a cardiac structure of a patient, measured by a wireless monitor. The measured signal waveform may be processed to generate waveform parameter data. The cardiac structure parameter may be estimated based on the waveform parameter data.

In some variations, a wireless monitoring system is provided, comprising a wireless monitor including a pressure sensor configured to measure pressure, and a wireless device comprising a processor configured to process the measured pressure to estimate a physiological parameter of the patient based on the measured pressure. In some variations, a wireless monitoring system is provided, comprising a wireless monitor including a pressure sensor configured to measure pressure. The wireless monitor may further include a processor configured to process the measured pressure and estimate a physiological parameter of the patient based on the measured pressure. In some variations, the physiological parameter of the patient may comprise one or more of blood velocity, blood flow, blood acceleration, motion of a valve leaflet, thickness of a valve leaflet, deterioration of a valve leaflet, paravalvular leak, circumferential extent, motion of a heart wall, thickness of a heart wall, size of a heart chamber, and the like.

In some variations, a wireless monitoring system is provided, comprising one or more wireless monitors that may be coupled to an expandable implantable device positioned in a cardiovascular vessel. The one or more wireless monitors may be configured to measure one or more of blood pressure and blood velocity. In some variations, the cardiovascular vessel may comprise one or more of a great vessel, an inflow/outflow tract of a ventricle, atrium or valve, a coronary artery, a peripheral artery, a peripheral vein, and the like. In some variations, the cardiovascular vessel may comprise one or more of a left ventricular outflow tract (LVOT) and a right ventricular outflow tract (RVOT). In some variations, the expandable implantable device may comprise one or more of a stent and a prosthetic heart valve.

In some variations, a wireless monitoring system is provided, comprising one or more wireless monitors that may be attached to an expandable structure. The expandable structure may be attached to an implantable device structure, and may be configured to avoid excessive force and/or pressure on the one or more wireless monitors during expansion of the implantable device structure. In some variations, the expandable structure may comprise one or more of a cuff, a ring, a mesh, a sheath, a ribbon, a thread and a suture. In some variations, the expandable structure may be made of a material that may comprise one or more of a stretchable, flexible, shock-absorbing, cushion-like, compressible, elastic, super-elastic, viscoelastic, hard, and a shape memory material.

In some variations, the wireless device may be configured to be disposed external and physically separate from the wireless monitor. In some variations, the wireless device may be configured to wirelessly power one or more wireless monitors. In some variations, the wireless device may be configured to transmit a downlink signal to the wireless monitor. In some variations, a first wireless monitor may be configured to transmit an uplink signal to one or more of a wireless device and a second wireless monitor. In some variations, the wireless monitor may be powered by an energy storage device comprising one or more of a rechargeable battery, a non-rechargeable battery, a capacitor, a super-capacitor, and the like. In some variations, the signal waveform may be generated by one or more of a wireless monitor and a wireless device. In some variations, the signal waveform may comprise one or more of ultrasonic, acoustic, vibrational, magnetic, electric, infrared (IR), optical, radiofrequency (RF), galvanic, and surface wave signals. In some variations, the signal waveform may comprise one or more of a continuous wave, a pulsed wave, and a modulated wave. In some variations, the signal waveform may comprise an ultrasonic signal with a carrier frequency of between about 0.1 MHz and about 100 MHz.

In some variations, the wireless monitor may be disposed within or on one or more of a cardiac structure and a vascular structure. In some variations, the wireless monitor may be coupled to an implantable device. In some variations, the wireless monitor may comprise a single transducer and a multiplexer.

In some variations, the wireless monitor may comprise memory to store one or more of waveform parameter data, physiological parameter data, patient data, wireless monitoring system data, and implantable device data. In some variations, the wireless monitor may comprise one or more of a pressure sensor, temperature sensor, electrical sensor, magnetic sensor, electromagnetic sensor, neural sensor, force sensor, flow or velocity sensor, acceleration sensor, chemical sensor, oxygen sensor, audio sensor, sensor for sensing other physiological parameters, and a stimulator.

In some variations, the implantable device may comprise one or more of prosthetic heart valves, prosthetic heart valve conduit, valve leaflet coaptation devices, annuloplasty rings, valve repair devices, septal occluders, appendage occluders, ventricular assist devices, pacemakers, implantable cardioverter defibrillators, cardiac resynchronization therapy devices, insertable cardiac monitors, stents, stent grafts, scaffolds, embolic protection devices, embolization coils, endovascular plugs, vascular patches, vascular closure devices, interatrial shunts, parachute devices for treating heart failure, cardiac loop recorders, and the like.

In some variations, the wireless device may comprise one or more of a wearable device, a handheld device, a probe connected to a measurement setup, a device placed on the patient's skin, a device not touching the patient, a laptop, a computer, a tablet, a mobile phone, a device permanently implanted in the body, a device temporarily implanted in the body, a communication device, combinations thereof, and the like.

### BRIEF DESCRIPTION OF THE DRAWINGS

FIG. 1 is a schematic block diagram of an illustrative variation of a wireless monitoring system.

FIG. 2 is a schematic block diagram of an illustrative variation of a wireless monitoring system comprising a wireless monitor with two transducers.

FIG. 3 is a schematic block diagram of an illustrative variation of a wireless monitor comprising a multiplexer comprising transmit/receive switches.

FIG. 4 is an illustrative view of a variation of a wireless monitoring system for Doppler measurement of blood velocity.

FIG. 5A is an illustrative top view, and FIG. 5B is an illustrative side view, of a variation of a wireless monitoring system for Doppler measurement of blood velocity.

FIG. 6 is an illustrative view of a set of wireless monitors implanted in a blood vessel.

FIG. 7 is an illustrative flowchart of a variation of a method of estimating blood velocity using pulse arrival time measurement.

FIG. 8 is an illustrative timing diagram for a variation of estimating blood velocity using pulse arrival time measurement.

FIG. 9A is an illustrative block diagram of a variation of a pulse detector. FIG. 9B is an illustrative block diagram of a variation of a pulse arrival time detector.

FIG. 10 is an illustrative flowchart of a variation of a method of estimating blood velocity using phase measurement.

FIG. 11A is an illustrative plot of a blood velocity waveform in the left ventricular outflow tract (LVOT). FIG. 11B is an illustrative plot of a variation of a received CW signal at a certain time. FIG. 11C is an illustrative plot of a variation of a received CW signal at another time. FIG. 11D is an illustrative plot of a phase shift of the received CW signal as a function of time.

FIG. 12 is an illustrative block diagram of a variation of a phase detector.

FIG. 13 is an illustrative flowchart of a variation of a method of estimating heart wall thickness using a wireless monitoring system.

FIG. 14A is an illustrative view of a variation of a wireless monitoring system used for estimating heart wall thickness. FIG. 14B is an illustrative timing diagram of a variation of signal waveforms used for estimating heart wall thickness. FIG. 14C is an illustrative plot of estimated heart wall thickness as a function of time over one or more cardiac cycles. FIG. 14D is an illustrative graph showing a variation of long-term tracking of an average heart wall thickness.

FIG. 15 is an illustrative view of a variation of a prosthetic aortic valve and wireless monitors.

FIG. 16 is an illustrative view of a variation of an implantable device and wireless monitors, implanted in the LVOT.

### DETAILED DESCRIPTION

Described here are systems, devices, and methods for monitoring one or more physiological characteristics or parameters of a patient. Generally, the systems described here may comprise one or more wireless monitors, which may be stand-alone or may be coupled to an implantable device, and one or more external wireless devices. The wireless monitor may measure a signal waveform transmitted at least through one or more of a fluid and a physiological structure of a patient, and one or more parameters of the signal waveform may be used to estimate a physiological parameter and/or assess the functionality of the implantable device. The wireless monitor, having a compact size and low power consumption, may be wirelessly powered by and in communication with an external wireless device. The wireless monitoring system described herein may be configured to monitor a fluid parameter (e.g., blood velocity), a property of a physiological structure (e.g., a heart wall), and/or performance of an implantable device (e.g., a CV implantable device). The signal waveform received or measured by a wireless monitor may be processed to generate waveform parameter data, and estimate a physiological parameter.

In some variations, one or more of the physiological parameters described herein may be used to diagnose, assess and/or monitor one or more of prosthetic valve operation, prosthetic valve dysfunction including obstruction and/or regurgitation (e.g., paravalvular, transvalvular, supra-skirtal), function of the ventricles (e.g., LV), atria (e.g., LA), or the heart, diagnosis or monitoring of heart failure, obstruction or restenosis in a stent and/or blood vessel, other cardiovascular diseases, other diseases, combinations thereof, and the like.

The measurement of a signal waveform and estimation of a physiological characteristic or parameter may be performed during predetermined intervals, continuously and/or on demand (e.g., by sending a wireless downlink command to a wireless monitor from an external wireless device). The results of the estimation may be output to one or more of the wireless monitor, external wireless device, computing device, dock, network, server, database, combinations thereof, and the like. Additionally (e.g., concurrently) or alternatively, the resultant blood velocity estimation may be output to one or more of a health care professional, technician, and designated users (e.g., patient, partner, family, support group).

### I. Systems

A. Overview

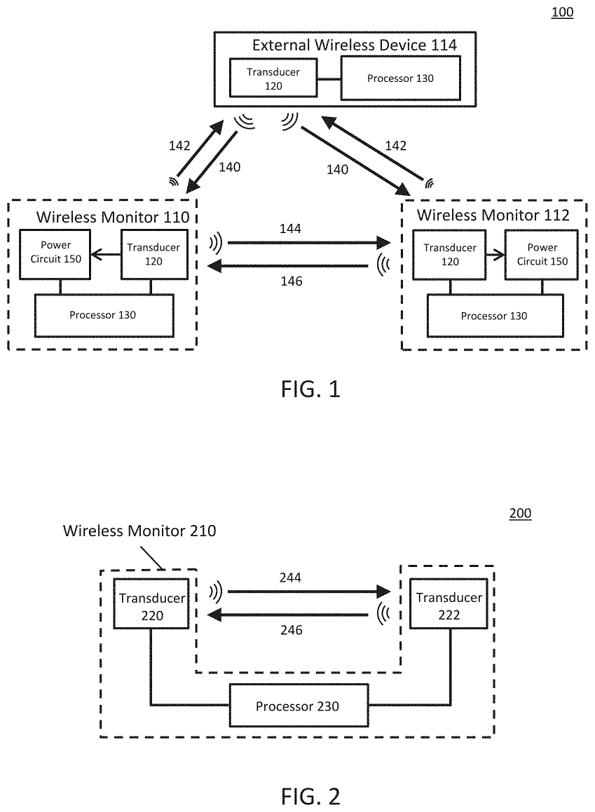

A wireless monitoring system may include one or more of the components necessary to estimate a physiological parameter based on a set of characteristics of a measured signal waveform and/or based on sensors, as described herein. FIG. 1 is an illustrative block diagram of a variation of a wireless monitoring system (100). The system (100) may comprise a first wireless monitor (110), a second wireless monitor (112), and an external wireless device (114). The wireless monitors (110, 112) may be wireless with respect to communication and/or power with another device. The wireless monitor (110, 112) may further receive and/or transmit, via one or more transducers (120) or sensors, signal waveforms traveling at least through one or more of a fluid and a physiological structure of the patient. In some variations, the wireless monitor (110, 112) may be a stand-alone implantable device, or may be coupled (e.g., attached) to another implantable device such as an implantable cardiac device (e.g., heart valve, stent).

In some variations, the external wireless device (114) may be configured to be disposed external and physically separate from at least the wireless monitor (110, 112). For example, the external wireless device (114) may be located external to a body of the patient. The external wireless device (114) may be configured to provide wireless power to one or more wireless monitors (110, 112), transmit data (e.g., digital data bits), and/or other signals to one or more wireless monitors (110, 112) using a downlink signal (140), and receive or measure data and/or other signals from one or more wireless monitors (110, 112) using an uplink signal (142).

In some variations, an uplink signal (142, 144) and downlink signal (140, 146) may be generated using one or more of mechanical waves (e.g., acoustic, ultrasonic, vibrational), magnetic fields (e.g., inductive), electric fields (e.g., capacitive), electromagnetic waves (e.g., RF, optical), galvanic coupling, surface waves, and the like. In some variations, the uplink signal (142, 144) may be an active data uplink or a passive data uplink (e.g., using backscatter). In some variations, a downlink signal (140) for data communication may be formed between an external wireless device (114) and wireless monitors (110, 112). For example, the external wireless device (114) may transmit to the wireless monitor (110, 112) one or more commands (e.g., measure blood velocity) and/or data (e.g., pulse repetition period, carrier frequency, computation data, data for programming the wireless monitor, and the like). In some variations, an uplink signal (144) may be formed between two wireless monitors (110, 112). In some variations, the downlink signal (140, 146) and/or the uplink signal (142, 144) may be modulated using any known digital or analog data modulation technique such as ASK, FSK, PSK, AM, FM, PM, pulse modulation, PAM, PWM, PPM, PCM, PDM, and the like.

In some variations, power, data and/or other signals may be transferred using the same energy modality (e.g., ultrasound). In some other variations, power, data and/or other signals may be transferred using different energy modalities. For example, the external wireless device (114) may provide wireless power to one or more wireless monitors (110, 112) using inductive power transfer and a signal (e.g., a signal waveform) may be transmitted from one wireless monitor (110) to another wireless monitor (112) using ultrasound.

In some variations, the wireless monitoring system (100) may comprise one wireless monitor (110) and one external wireless device (114). In some variations, a signal waveform may be transmitted by one or more of the external wireless device (114) and the wireless monitor (110), and the signal waveform may be received or measured by the wireless monitor (110). In some variations, one or more wireless monitors (110, 112), instead of or in addition to being configured for transmitting and/or receiving a signal waveform, may comprise one or more sensors (e.g., a pressure sensor) that may measure a physiological parameter of a patient (e.g., blood pressure, pressure inside the heart wall).

FIG. 2 shows an illustrative variation of a wireless monitor (210) that may be used in some variations of the wireless monitoring system (200) described herein. An external wireless device is not shown for simplicity. The wireless monitor (210) may comprise two or more transducers (220, 222), wherein the transducers may be configured to transmit and/or receive a signal waveform traveling at least through one or more of a fluid and a physiological structure of the patient.

B. Signal Waveform

Generally, the signal waveforms described here may be configured to wirelessly propagate through one or more of fluid and a physiological structure of a patient for measuring a physiological parameter of the patient. In some variations, the signal waveform may be transmitted by a wireless monitor (110, 112) through one or more transducers of the wireless monitor. In some variations, the signal waveform may be transmitted by the external wireless device (114). The signal waveform may be received or measured by one or more wireless monitors (110, 112) through one or more transducers of the wireless monitor.

In some variations, a signal waveform may be generated using one or more of mechanical waves (e.g., acoustic, ultrasonic, vibrational), magnetic fields (e.g., inductive), electric fields (e.g., capacitive), electromagnetic waves (e.g., RF, optical), galvanic coupling, surface waves, and the like. In some variations, a signal waveform may be generated in the form of a continuous wave (CW) signal or a pulsed wave (PW) signal. In some variations, the signal waveform may be generated using any known digital or analog modulation technique such as ASK, FSK, PSK, AM, FM, PM, pulse modulation, PAM, PWM, PPM, PCM, PDM, and the like. In some variations, an ultrasonic signal waveform may comprise a frequency of between about 0.1 MHz and about 100 MHz.

In some variations, a processor may process the measured signal waveform to generate waveform parameter data. Waveform parameter data may include one or more of a Doppler shift, a frequency shift, a phase shift, a time delay, pulse arrival time, phase, amplitude, frequency, pulse repetition period, pulse transit time, pulse duration, number of pulses, and the like, as described in more detail herein.

C. Fluid and Physiological Structure of a Patient

A fluid described here may include one or more of blood, plasma, urine and other bodily fluids. A physiological structure described here may include one or more of a cardiac structure, a vascular structure, a structure of a cardiovascular implantable device, and other biological and/or implantable device structures in the body.

In some variations, a physiological structure may comprise a cardiac structure. Generally, a cardiac structure may include one or more of an anatomical structure of the heart and material build up in the heart. An anatomical structure of the heart may include one or more of cardiac tissue, heart wall, heart muscle, heart chamber, ventricle, atrium, septum, heart valve, heart valve leaflet, chordae tendineae, aortic sinus, sinotubular junction, and the like. Material build up in the heart may include one or more of calcification, blood clot formation, thrombosis, endothelialization, endocarditis, infection, vegetation, pannus, scar tissue growth, healthy tissue growth, and the like.

In some variations, a physiological structure may comprise a vascular structure. Generally, a vascular structure may include one or more of an anatomical structure of the vasculature and material build up in the vasculature. An anatomical structure of the vasculature may include one or more of a cardiovascular vessel or blood vessel (e.g., artery, vein, capillary) such as one or more of great vessels (e.g., aorta, pulmonary artery), peripheral arteries and/or veins, coronary artery, superficial femoral artery, inflow and/or outflow tract of a ventricle and/or atrium (e.g., LVOT, RVOT), inflow and/or outflow regions of a valve, and the like, lumen, a wall of a blood vessel, and the like. Material build up in the vasculature may include one or more of plaque formation, fat deposits, blood clot formation, thrombosis, endothelialization, scar tissue growth, healthy tissue growth, and the like.

In some variations, a physiological structure may comprise one or more of a structure of a cardiovascular implantable device and material build up on a cardiovascular implantable device. A cardiovascular implantable device may be implanted permanently or temporarily in the body. A structure of a cardiovascular implantable device may include one or more of prosthetic valve leaflets or cusps, prosthetic valve commissures, mechanical valve discs, cage or ball, stent structure of a stent device or prosthetic heart valve, and the like. Material build up on a cardiovascular implantable device may include one or more of material build up on prosthetic heart valves (e.g., calcification, thrombus formation, and the like, on a prosthetic valve leaflet; endothelialization, calcification, and the like, on a stent structure of a transcatheter heart valve, and so on), material build up on a stent (e.g., endothelialization, plaque formation, scar tissue growth), and the like.

D. Physiological Parameter of a Patient

Generally, a physiological parameter of a patient may include one or more of a cardiac parameter, a cardiac structure parameter, a vascular structure parameter, a parameter of the structure of a cardiovascular implantable device, a biological parameter, a parameter of an implantable device, and the like.

In some variations, a physiological parameter may comprise a cardiac parameter. Generally, a cardiac parameter may include one or more of a parameter related to blood, flow of blood, and the functioning of the heart and/or its components. As used herein, blood flow or flow of blood may refer to the motion of blood and any physical property of flowing blood, in general, unless specified otherwise. In certain instances, blood flow may refer specifically to the volume flow of blood per unit time (e.g., measured in mL/s). In other instances, blood flow may refer, in general, to one or more of blood velocity, turbulence, acceleration, combinations thereof, and the like. A cardiac parameter may comprise one or more of blood velocity, blood flow, peak velocity, mean velocity, blood velocity as a function of time, blood velocity in the LVOT, blood velocity in the RVOT, aortic blood velocity, blood velocity and/or flow in the pulmonary artery, velocity-time integral (VTI), a Doppler velocity index or dimensionless velocity index (DVI), turbulence, acceleration, blood pressure, blood pressure waveform as a function of time, blood temperature, pressure gradient across a valve, mean gradient, peak gradient, stroke volume, cardiac output, heart rate, ECG, EKG, aortic regurgitation (AR) index and other regurgitation parameters, valve stenosis parameters, pressure volume loops, any parameters related to heart valve function, combinations thereof, and the like.

In some variations, a physiological parameter may comprise a cardiac structure parameter. Cardiac structure parameters may comprise one or more of heart wall thickness, heart wall motion, pressure inside the heart muscle or wall, heart wall contractility, force of contraction of heart muscle, a mechanical property of the heart wall (e.g., hardness, stiffness, density), oxygen content, myocardial oxygen consumption, ventricular volume or size, pressure volume loops, ventricle mass, ventricle function, ventricular ejection fraction, effective cross-sectional area of a valve orifice or effective orifice area (EOA), cross-sectional area of ventricular outflow tract, endothelial tissue thickness, calcification thickness, any parameters related to heart valve structures, any parameters related to heart failure, any other property of a cardiac structure (e.g., mechanical, physical, chemical, biological), a property of material build up on a cardiac structure, combinations thereof, and the like.

In some variations, a physiological parameter may comprise a vascular structure parameter. Vascular structure parameters may comprise one or more of blood vessel wall thickness, blood vessel wall motion, plaque thickness, endothelial tissue thickness, blood vessel lumen diameter, any parameters related to blood vessels, any parameters related to heart failure, any other property of a vascular structure (e.g., mechanical, physical, chemical, biological), a property of material build up on a vascular structure (e.g., plaque thickness), combinations thereof, and the like.

In some variations, a physiological parameter may comprise a parameter of a structure of a cardiovascular implantable device. Parameters of a structure of a cardiovascular implantable device may comprise one or more of prosthetic valve (PV) leaflet thickness, PV leaflet motion, mechanical property of a PV leaflet (e.g., hardness, stiffness), PV leaflet calcification, acoustic property of a PV leaflet, size of a PV leaflet opening, circumferential extent (CE), effective regurgitant orifice area (EROA), regurgitant volume (RV), regurgitant fraction (RF), any parameters related to one or more of PV obstruction or stenosis, PV regurgitation, PV endocarditis, thrombosis, pannus, patient-prosthesis mismatch, stent re-stenosis, any parameter related to material build up on a cardiovascular implantable device, combinations thereof, and the like.

E. Wireless Monitor

Generally, the wireless monitors described here may be configured to measure a signal waveform transmitted through one or more of a fluid and a physiological structure of a patient. In some variations, the signal waveform may be processed to generate waveform parameter data used to estimate a physiological parameter of the patient as described herein. In some variations, the wireless monitor may be controlled from an external wireless device or another wireless monitor. In some variations, the wireless monitors described here may be configured to perform only a subset of the measurement, processing, and estimation steps described herein. In some variations, the wireless monitors may comprise only a subset of the components described herein.

In some variations, the wireless monitor (110, 112) may be stand-alone and implanted in the body. In some variations, the wireless monitor (110, 112) may be disposed within or on one or more of a cardiac structure (e.g., heart valve, heart chamber), a vascular structure (e.g., pulmonary artery, any other blood vessel), and the like. In some variations, the wireless monitor (110, 112) may be coupled (e.g., attached) to an implantable device (e.g., a prosthetic heart valve, a stent, and the like) and/or an expandable structure.

In some variations, the wireless monitor (110, 112) may comprise a transducer (120) configured to transmit and/or receive or measure a signal waveform, a processor (130) configured to process the measured signal waveform and/or to control the wireless monitor (110, 112), and a power circuit (150) configured to recover and condition received wireless power and thereby provide power to operate the wireless monitor (110, 112). In some variations, wireless power may be transmitted by the external wireless device (114) via a downlink signal (140) and received by the transducer (120) of a wireless monitor (110, 112). In some variations, the wireless monitor (110, 112) may be powered by one or more energy storage devices. In some variations, one or more wireless monitors (110, 112) may be partially or fully powered through energy harvesting techniques and/or by wireless power provided by another wireless monitor or device implanted in a patient.

a. Transducer

Generally, the transducer described here may be configured to convert between a wireless energy modality and electrical signals. The wireless energy modality may comprise one or more of mechanical waves (e.g., acoustic, ultrasonic, vibrational), magnetic fields (e.g., inductive), electric fields (e.g., capacitive), electromagnetic waves (e.g., RF, optical), galvanic coupling, surface waves, combinations thereof, and the like. The transducer may be configured to perform one or more of receiving wireless power, receiving wireless data and/or other signals (e.g., a signal waveform), transmitting wireless data and/or other signals (e.g., a signal waveform), and the like.

In some variations, the transducer (120) may comprise one or more of an ultrasonic transducer, a radiofrequency (RF) transducer (e.g., a coil, an RF antenna), a capacitive transducer, combinations thereof, and the like. In some variations, an ultrasonic transducer may comprise one or more of a piezoelectric device, a capacitive micromachined ultrasonic transducer (CMUT), a piezoelectric micromachined ultrasonic transducer (PMUT), combinations thereof, and the like. In some variations, an ultrasonic transducer may be configured to convert pressure and/or force into an electrical signal, and/or vice versa. In some variations, the transducer (120) may comprise one or more ultrasonic transducers that may be of one or more types, including but not limited to, piston (e.g., rod, plate), cylindrical, ring, spherical (e.g., shell), flexural (e.g., bar, diaphragm), flextensional, combinations thereof, and the like. In some variations, a piezoelectric device may be made of one or more of lead zirconate titanate (PZT), PMN-PT, Barium titanate (BaTiO3), PVDF, Lithium niobate (LiNbO3), any derivates thereof, and the like.

In some variations, an ultrasonic transducer may be configured to operate at a frequency between about 20 kHz to about 20 MHz for receiving power from an external wireless device. Operation in such a frequency range may be useful to miniaturize the ultrasonic transducer to millimeter or sub-millimeter dimensions, which may allow integration of one or more wireless monitors on an implantable device such as a stent.

In some variations, the transducer (120) may comprise a single transducer element (e.g., ultrasonic piezoelectric device) that may allow miniaturization of the wireless monitor, which may be advantageous for integrating the wireless monitor onto other devices such as prosthetic valves or stents. The wireless monitor may comprise a multiplexer (as described later) to allow the single transducer element to perform one or more functions of a transducer as described above.

In some variations, the transducer (120) may comprise a plurality of transducer elements or one or more arrays of transducer elements, wherein the transducer elements may be of the same type (e.g., ultrasonic) or different types (e.g., ultrasonic, RF, infrared), and may be configured to perform one or more functions of the transducer. For example, a first transducer element may comprise an RF coil configured to receive power and communicate data and/or other signals with at least the external wireless device. A second transducer element may comprise an ultrasonic transducer configured to transmit and/or receive a signal waveform into/from blood for estimation of blood velocity. In some variations, a transducer may have a volume of less than about 10 cm³. This small size may allow one or more wireless monitors to be attached to an implantable device such as a cardiac implantable device (e.g., prosthetic heart valve), and/or may allow minimally invasive delivery of the wireless monitor into the body (e.g., via percutaneous or transcatheter techniques).

In some variations, one or more transducers (e.g., an ultrasonic transducer) or transducer elements of one or more wireless monitors may be oriented or tilted towards one or more of a target location, a transducer of another wireless monitor, a transducer of the same wireless monitor, a transducer of the external wireless device, combinations thereof, and the like. This may help with preferably aligning the radiation pattern of a transducer for a given measurement technique described herein, to increase the received signal or signal-to-noise ratio, thereby, allowing reliable and accurate estimation of a physiological parameter and/or communication of data.

In some variations, an ultrasonic transducer (120) of a wireless monitor may comprise more than one ultrasonic transducer elements configured to achieve a combined or overall radiation pattern with a wide acceptance angle or −3 dB beam width. This may be advantageous when a signal waveform may need to be transmitted and/or received from a large angle relative to a main lobe of a given ultrasonic transducer element's radiation pattern. In some variations, one or more ultrasonic transducer elements of the wireless monitor may have a different feature or a property with respect to each other that may allow the combined assembly of the one or more ultrasonic transducer elements to have a wide acceptance angle or beam width. In some variations, such a different feature or property may comprise one or more of the following, including but not limited to, a position or an orientation or an angle in which a transducer element may be assembled relative to other elements (e.g., on a flat substrate or mounted on a specific structure that may allow assembling elements at different angles with respect to each other, and the like), dimensions of a transducer element, material of a transducer element, poling direction of a piezoelectric element, poling direction relative to the electrode locations (e.g., side-electroded structures), combinations thereof, and the like. For example, in some variations, an ultrasonic transducer (120) may comprise three ultrasonic transducer elements that may be oriented at a non-zero angle (e.g., orthogonally, at an angle of 30°, and the like) relative to each other.

In some variations, each wireless monitor may comprise one or more transducers. In some variations, two or more wireless monitors may share one or more transducers. For example, a stent device may comprise an RF coil with two or more feeds or ports, to which two or more wireless monitors may be connected. In some variations, two or more wireless monitors may be connected in parallel to a single feed or port of a transducer.

b. Power Circuit

Generally, the power circuits described here may be configured to recover, condition and/or control wireless power received by a transducer. For example, a power circuit may be configured to receive electrical power from a transducer and convert the received power into usable energy for powering one or more circuit blocks, and/or recharging one or more energy storage devices, of a wireless monitor. In some variations, a power circuit (150) may comprise one or more of a rectifier, a voltage regulator, voltage/current reference circuits, one or more energy storage elements or devices (e.g., rechargeable battery, non-rechargeable battery, capacitor, super-capacitor), and the like. In some variations, the power circuit (150) may comprise a rechargeable battery for energy storage, along with a capacitor in parallel with the battery, wherein the capacitor may sink and/or supply at least a part of the current during one or more functions of the wireless monitor. In some variations, the power circuit may not include an energy storage device (e.g., to reduce size of the wireless monitor), and the wireless monitor may be wirelessly powered by another device concurrently while executing its functions.

In some variations, the systems, devices, and methods disclosed herein may comprise one or more systems, devices, and methods described in U.S. Pat. No. 9,544,068, filed on May 13, 2014, U.S. Pat. No. 10,177,606, filed on Sep. 30, 2016, and U.S. Pat. No. 10,014,570, filed on Dec. 7, 2016, the contents of each of which are hereby incorporated by reference in its entirety.

c. Multiplexer

Generally, the multiplexer described here may be configured to decouple one or more of power signal, data signal and other signals in a wireless monitor. This may be done in order to avoid interference between these signals and ensure proper functioning of the wireless monitor. In some variations, a multiplexer may enable using a single transducer for several functions such as receiving wireless power/data, transmitting wireless data, receiving a signal waveform, transmitting a signal waveform, combinations thereof, and the like. For example, a multiplexer in a wireless monitor may be configured to decouple a power signal from a data signal received from an external wireless device such that the power signal is provided to the power circuit for power recovery and conditioning, and the data signal is provided to the processor for data recovery. In some variations, a wireless monitor may be configured to perform a Doppler measurement of blood velocity, wherein the wireless monitor may comprise a multiplexer, and a single ultrasound transducer for one or more of power recovery, data communication (uplink, downlink), transmitting and/or receiving signal waveform(s) for Doppler measurements, combinations thereof, and the like.

In some variations, the multiplexer may comprise one or more of transmit/receive switches, passive devices (e.g., diodes, relays, MEMS circuits, blockers, passive switches), circulators, frequency selection (e.g., using filters, impedance matching networks), direct wired connections, combinations thereof, and the like.

In some variations, the transmit/receive switches may be driven based on timing control or time multiplexing such that one or more of power signal, data signal and other signals are received by a wireless monitor at different times. In some variations, the transmit/receive switches may be driven based on amplitude selection wherein one or more of power signal, data signal and other signals have different amplitudes. In some variations, the transmit/receive switches may be driven based on frequency selection or frequency multiplexing wherein one or more of power signal, data signal and other signals have different frequencies. In some variations, the transmit/receive switches may be implemented using depletion-mode transistors to operate when the wireless monitor may not have power, stored energy or an established voltage rail.

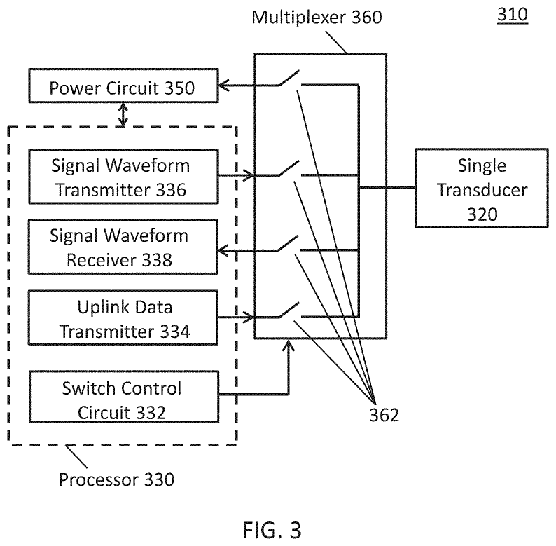

FIG. 3 is an illustrative block diagram of a variation of a wireless monitor (310) comprising a single transducer (320) and a multiplexer (360) comprising transmit/receive switches (362). The multiplexer (360) may be interfaced with a power circuit (350) and a processor (330). The processor (330) may comprise a switch control circuit (332) to control the turning on/off of the switches (362) in the multiplexer (360). As shown in FIG. 3, the processor (330) may also comprise an uplink data transmitter (334), a signal waveform transmitter (336) and a signal waveform receiver (338).

d. Processor

Generally, the processor described here may receive, transmit and/or process data and/or other signals, and/or control one or more components of the system (e.g., wireless monitor). The processor may be configured to receive, process, compile, compute, store, access, read, write, and/or transmit data and/or other signals (e.g., a signal waveform). Additionally, or alternatively, the processor may be configured to control one or more components such as a multiplexer, a transducer, a sensor, and the like. One or more processors, as described herein, may be included in one or more of a wireless monitor, an external wireless device, and the like.

In some variations, the processor may be configured to access or receive data and/or other signals from one or more of a transducer, a sensor (e.g., pressure sensor) and a storage medium (e.g., memory). For example, the processor may comprise one or more of a signal waveform receiver, a downlink data receiver, an envelope detector circuit, an amplifier (e.g., a low-noise amplifier or LNA), a filter, a frequency detector circuit, a phase detector circuit, comparator circuits, decoder circuits, combinations thereof, and the like.

In some variations, the processor may be any suitable processing device configured to run and/or execute a set of instructions or code (e.g., DSP, graphics processing unit, machine learning processor, and the like). The systems, devices, and/or methods described herein may be performed by software (executed on hardware), hardware, or a combination thereof. Software modules (executed on hardware) may be expressed in a variety of software languages (e.g., computer code).

In some variations, the processor may be configured to process a signal waveform to generate waveform parameter data. For example, a processor may comprise one or more of a frequency detector, phase detector, a Fast Fourier Transform (FFT) circuit, time-to-digital converter (TDC) circuit, an integrator circuit, a sampling circuit, an analog-to-digital converter (ADC) circuit, a timer circuit, a clock, a counter, an oscillator, a phase-locked loop (PLL), a frequency locked loop (FLL), combinations thereof, and the like.

In some variations, the processor may be configured to estimate a physiological parameter of the patient (e.g., blood velocity) based on the waveform parameter data (e.g., a set of pulse arrival times). For example, a processor may comprise a difference amplifier (e.g., subtractor), a digital signal processor (DSP), an integrator, an adder circuit, a multiplier circuit, a finite state machine, combinations thereof, and the like.

In some variations, the processor may be configured to generate or transmit data and/or other signals through one or more of a transducer, a storage medium, and the like. For example, a processor of a wireless monitor may comprise one or more of a signal waveform transmitter, an uplink data transmitter, an oscillator, a power amplifier, a mixer, an impedance matching circuit, a switch, a driver circuit, combinations thereof, and the like.

In some variations, the processor may be configured to control one or more blocks in a wireless monitor and/or an external wireless device. For example, the processor may be configured to control the operation of a multiplexer in a wireless monitor (e.g., turning on/off of the transmit/receive switches in a multiplexer).

In some variations, a first processor may be included in a wireless monitor and a second processor may be included in an external wireless device. In such variations, a wireless monitor may be configured to measure a signal waveform, the first processor may be configured to generate waveform parameter data, and the second processor may be configured to estimate a physiological parameter of the patient based on the waveform parameter data. In some variations, the first processor itself may be configured to also estimate a physiological parameter of the patient based on the waveform parameter data.

e. Memory

Generally, the wireless monitor and/or the external wireless device described here may comprise a memory configured to store data and/or information temporarily or permanently. In some variations, the memory may be of one or more types, including but not limited to, random access memory (RAM), static RAM (SRAM), dynamic RAM (DRAM), resistive random-access memory (ReRAIVI or RRAM), magnetoresistive random-access memory (MRAM), ferroelectric random-access memory (FRAM), standard-cell based memory (SCM), shift registers, read-only memory (ROM), programmable read-only memory (PROM), erasable programmable read-only memory (EPROM), electrically erasable programmable read-only memory (EEPROM), flash memory (e.g., NOR, NAND), embedded flash, volatile memory, non-volatile memory, one time programmable (OTP) memory, combinations thereof, and the like.

In some variations, the memory may store instructions to cause the processor to execute modules, processes, and/or any functions associated with a wireless monitor and/or an external wireless device. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) may be non-transitory in the sense that it may not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In some variations, the memory may be configured to store any received data and/or data generated by a wireless monitor and/or an external wireless device. In some variations, the memory (e.g., of a wireless monitor and/or of an external wireless device) may store one or more of waveform parameter data, one or more parameters required to process a signal waveform to generate waveform parameter data, physiological parameter data (e.g., values of cardiac parameters, cardiac structure parameters, vascular structure parameters, and the like), one or more parameters required to estimate a physiological parameter of a patient from waveform parameter data, patient data (e.g., diagnosis information, surgery or procedure data, prosthetic valve baseline and/or follow up data, imaging data such as echocardiography images, blood flow/pressure data, and the like), wireless monitoring system data (e.g., number and/or location of wireless monitors, one or more identification (ID) numbers corresponding to one or more wireless monitors, speed of sound in blood and/or tissue, and the like), implantable device data (e.g., type, size, position of a prosthetic valve, and the like), combinations and derivatives thereof, and the like.

f. Sensor

Generally, the sensors described here may be configured to sense, receive and/or transmit a signal corresponding to one or more parameters. In some variations, the sensor may comprise one or more of a transducer, pressure sensor, temperature sensor, electrical sensor (e.g., impedance sensors), magnetic sensor, electromagnetic sensor (e.g., infrared photodiode, optical photodiode, RF antenna), neural sensor (e.g., for sensing neural action potentials), force sensor, flow or velocity sensor, acceleration sensor, chemical sensor (e.g., pH sensor, protein sensor, glucose sensor), oxygen sensor (e.g., pulse oximetry sensor, myocardial oxygen consumption sensor), audio sensor (e.g., a microphone to detect heart murmurs, prosthetic valve murmurs, auscultation), sensor for sensing other physiological parameters (e.g., sensors to sense heart rate, breathing rate, arrhythmia, motion of heart walls), a stimulator (e.g., for stimulation and/or pacing function), combinations thereof, and the like.

In some variations, one or more pressure sensors may be used for one or more of monitoring heart function and/or heart failure (e.g., measuring pressure in the LV, RV, LA, RA, pulmonary artery, aorta, and the like), monitoring a prosthetic valve (e.g., valve pressure gradients to monitor stenosis), monitoring a stent device (e.g., measuring pressure in the lumen), estimation and/or verification of blood velocity measurements (e.g., using the Bernoulli equation), combinations thereof, and the like. In some variations, one or more pressure sensors may be of the following types, including but not limited to, an absolute pressure sensor, a gauge pressure sensor, a sealed pressure sensor, a differential pressure sensor, an atmospheric pressure sensor, combinations thereof, and the like. In some variations, one or more pressure sensors may be based upon one or more pressure-sensing technologies, including but not limited to, resistive (e.g., piezoresistive, using a strain gauge or a membrane to create a pressure-sensitive resistance, and the like), capacitive (e.g., using a diaphragm or a membrane to create a pressure-sensitive capacitance, and the like), piezoelectric, optical, resonant (e.g., pressure-sensitive resonance frequency of a structure, and the like), combinations thereof, and the like. In some variations, a pressure sensor may be manufactured using Micro-Electro-Mechanical Systems (MEMS) technology. In some variations, a pressure sensor may comprise one or more of a stagnation pressure sensor, a static pressure sensor, and the like.

In some variations, the sensor may comprise a stimulator used for stimulating the muscles and/or neurons or nerves of one or more of cardiac tissue (e.g., HIS bundle, atrioventricular node), heart chamber (e.g., septal, lateral walls of the LV), blood vessel wall, combinations thereof, and the like. For example, one or more stimulators may be used to stimulate the LV wall for pacing and/or cardiac resynchronization. In some variations, a stimulator may comprise an electrical stimulator (e.g., electrodes), an ultrasonic stimulator (e.g., ultrasonic transducer), an optical stimulator (e.g., an optical LED), an infrared stimulator (e.g., an infrared LED), a thermal stimulator (e.g., electrodes to generate heat in tissue), combinations thereof, and the like.

F. Implantable Device

Generally, the implantable devices described here may be configured to be implanted inside a patient or an animal. One or more wireless monitors may be coupled (e.g., attached) to one or more of an implantable device, any part of an implantable device, an expandable structure, and the like. In some variations, an implantable device may comprise one or more of prosthetic heart valves, prosthetic heart valve conduit, valve leaflet coaptation devices, annuloplasty rings, valve repair devices (e.g., clips, pledgets), septal occluders, appendage occluders, ventricular assist devices, pacemakers (e.g., including leads, pulse generator), implantable cardioverter defibrillators (e.g., including leads, pulse generator), cardiac resynchronization therapy devices (e.g., including leads, pulse generator), insertable cardiac monitors, stents (e.g., coronary or peripheral stents, fabric stents, metal stents), stent grafts, scaffolds, embolic protection devices, embolization coils, endovascular plugs, vascular patches, vascular closure devices, interatrial shunts, parachute devices for treating heart failure, cardiac loop recorders, combinations thereof, and the like. For example, a prosthetic heart valve may comprise one or more of a transcatheter heart valve (THV), self-expandable THV, balloon-expandable THV, surgical bioprosthetic heart valve, mechanical valve, and the like.

Generally, the implantable devices described here may be located in or near any region in the body, including but not limited to heart valves (e.g., aortic valve, mitral valve), heart chambers (e.g., LV, RV, LA, RA), blood vessels (e.g., pulmonary artery, aorta, superficial femoral artery, coronary artery, pulmonary vein, and the like), heart tissue (e.g., heart muscle or wall, septum), gastrointestinal tract (e.g., stomach, esophagus), bladder, combinations thereof, and the like.

a. Methods of Integrating Wireless Monitors on Implantable Devices

Wireless monitors or sensors may need to be integrated on implantable devices that undergo a physical expansion during implantation or during their operation. For instance, miniature wireless monitors or sensors may need to be integrated on implantable devices such as transcatheter heart valves, stents, and the like. Such implantable devices may first be in a crimped state and may then be expanded during delivery to a target location in the body. Wireless monitors directly attached to a crimped implantable device may experience a large force or pressure during device expansion, which may crush or damage the wireless monitors. Solutions provided herein may help with mitigating this challenge.

In some variations, one or more wireless monitors may be attached to an expandable structure, wherein the expandable structure may be attached at one or more locations (e.g., on the outside, or inside) of an implantable device structure. The expandable structure may be configured to expand during expansion of the implantable device and help with avoiding excessive force or pressure on the wireless monitors. In some variations, the expandable structure may be in the form of a cuff, a ring, a mesh, a sheath, a ribbon, a thread, a suture, combinations thereof, and the like. In some variations, the expandable structure may be made of a material that may be stretchable (e.g., rubber, silicone), flexible, shock-absorbing, cushion-like, compressible, elastic, super-elastic (e.g., nitinol), viscoelastic, shape memory material, hard/stiff (e.g., materials like titanium, glass, and the like, that may be used to seal a wireless monitor and may withstand expansion forces, thereby protecting wireless monitors from damage), combinations thereof, and the like. For example, wireless monitors may be attached to a ring or a mesh made of an elastic material such as nitinol or rubber, and the ring or mesh may be wrapped fully or partially around a crimped prosthetic valve, or sewn/tied to it. When the crimped valve structure is expanded during deployment, the expansion of the ring/mesh may help prevent excessive force on the wireless monitor by absorbing the impact/shock.

In some variations, one or more wireless monitors may be attached to one or more locations on an expanding implantable device that do not undergo excessive expansion, or do not experience excessive force during expansion. For example, wireless monitors or sensors may be attached at the tips, ends or peaks of a THV stent, or to an extension that may be attached to a tip, end or a peak of a stent strut. During radial expansion of the THV, such locations may not experience excessive stretching, thus, avoiding damage to wireless monitors attached thereon.

G. Wireless Device

Generally, as used herein, wireless device or external wireless device may refer to any device that may be physically separate from one or more wireless monitors. In some variations, the external wireless device (114) may comprise a transducer (120) and a processor (130), as illustrated in FIG. 1. Different variations of the transducer (120) and the processor (130), as explained before in the context of the wireless monitor, are also applicable herein.

In some variations, an external wireless device may perform one or more functions, including but not limited to, transmitting one or more of wireless power, data and other signals to one or more wireless monitors, receiving one or more of wireless data and other signals from one or more wireless monitors, processing data and/or signals (e.g., estimating blood velocity from waveform parameter data, performing computations), performing sensing and/or actuation (e.g., measuring heart rate, ECG, EKG), storing data or information in memory, communicating with other external wireless devices, displaying data or information (e.g., on a screen or a monitor), generating alerts (e.g., visual, audio) for a user, combinations thereof, and the like.

In some variations, the external wireless device (114) may provide power and/or exchange data and/or other signals with one or more wireless monitors (110, 112) using one or more of mechanical waves (e.g., acoustic, ultrasonic, vibrational), magnetic fields (e.g., inductive), electric fields (e.g., capacitive), electromagnetic waves (e.g., RF, optical), galvanic coupling, surface waves, and the like.

In some variations, the external wireless device may be located at one or more locations, including but not limited to, outside the body (e.g., as a wearable device, a handheld device, a probe connected to a measurement setup, a device placed on skin, a device not touching the patient, a laptop, a computer, a mobile phone, a smart watch, and the like), permanently implanted inside the body (e.g., implanted under the skin, along the outer wall of an organ), temporarily implanted inside the body (e.g., located on a catheter or a probe inserted through a blood vessel, esophagus or the chest wall, used during surgery or procedure), combinations thereof, and the like. In some variations, an external wireless device placed on the body of a patient (e.g., placed on the chest) may communicate waveform parameter data received from one or more wireless monitors to another external wireless device such as a laptop, a tablet, a cell phone, and the like, which may process this waveform parameter data to estimate a physiological parameter of a patient. Such communication may or may not be done in real time as the data is generated. One or more processors that may process the waveform parameter data may be located in the same room or building as the wireless monitor and/or located remotely (e.g., in a different building, city, country).

In some variations, the external wireless device may further comprise a communication device configured to permit a user and/or health care professional to control and/or interact with one or more of the devices of the wireless monitoring system. The communication device may comprise a network interface configured to connect the external wireless device to another system (e.g., Internet, remote server, database) by wired or wireless connection. The communication device may further comprise a user interface, comprising one or more input and/or output devices. A user may comprise one or more of a subject or patient, a predetermined contact such as a partner, family member, health care professional, and the like. For example, an output device of the user interface may output one or more of a physiological parameter of a patient (e.g., blood pressure estimates, blood velocity), waveform parameter data, system data, alarms and/or notifications, combinations thereof, and the like. Input and/or output devices may comprise one or more of a display device, an audio device, a haptic/touch device, combinations thereof, and the like.

In some variations, the systems and methods described herein may be in communication with other wireless devices via, for example, one or more networks, each of which may be any type of network (e.g., wired network, wireless network).

### II. Methods

Also described here are methods for monitoring a physiological characteristic of a patient using the systems and devices described herein. In particular, the systems, devices, and methods described herein may be used to accurately estimate and track values of a physiological parameter of a patient, such as, for example, blood velocity.

In some variations, methods described here may include one or more of transmitting a signal waveform into a fluid or a physiological structure of a patient, receiving a corresponding signal waveform, processing the signal waveform to generate waveform parameter data, and estimating a physiological parameter of a patient based on the waveform parameter data. In some variations, methods described here may include estimating a physiological parameter of a patient based on one or more sensors such as pressure sensors.

A. Estimating Blood Velocity

Generally, the methods described here may include estimation of fluid velocity (e.g., blood velocity) using a wireless monitoring system. a. Doppler Measurements Using Implantable Wireless Monitor(s)

Blood velocity or flow is typically measured using Doppler ultrasonography. This technique uses an external imager (e.g., an ultrasound imaging probe) to measure the Doppler frequency and/or phase shift of an ultrasound signal reflected from moving blood cells. It typically needs a skilled sonographer, suffers from operator dependence, is resource-intensive and cannot be used for at-home monitoring of patients. Measuring flow using miniature implantable wireless monitors (WM) may overcome these drawbacks, but may come with new technical challenges. While an external imager can be freely repositioned by an operator in order to align the transmitted/received ultrasound waves parallel to the direction of flow, it may not be possible to reposition implantable WMs during a measurement. Alignment parallel to flow may be important because the measured Doppler shift may be proportional to νcos θ, where ν is the blood velocity and θ is the angle between the ultrasound wave and the direction of moving blood cells. Here, larger θ leads to a smaller Doppler shift, and erroneous results if θ is not precisely known. Further, as opposed to external imagers, miniature implantable WMs are limited in size, power and computational resources, thereby, making blood flow measurements challenging. Solutions presented herein may be useful to overcome these challenges.

A wireless monitoring system for flow measurements may comprise one or more WMs positioned in or near a vessel. A vessel may be any structure that supports fluid flow, such as great vessels, arteries, veins, capillaries, cardiac chambers, outflow/inflow tracts of cardiac chambers, and the like. Fluid may comprise one or more of blood, urine, water, saline, and the like. In some variations, a WM may comprise one or more transducers (or sensors), such as ultrasound transducers, wherein a transducer may be configured to transmit a signal waveform at least in part towards flowing blood, or receive a reflected signal waveform that is reflected at least in part from flowing blood, or both.

In some variations, an implantable WM may comprise one or more transducers positioned substantially in the center of the lumen of a vessel, or away from vessel walls, and oriented to transmit/receive signal waveforms substantially parallel to flow (e.g., θ<20°). Such orientation may be made possible by deploying the one or more transducers at junctions or forks where a vessel branches out, on a beam or a strut protruding into the vessel lumen (e.g., a narrow strut attached to a stent and designed to protrude into the lumen), attached to an implantable device deployed in the lumen of a vessel, and the like.

While the above solution may be practical for vessels with large diameters (e.g., aorta, pulmonary artery, etc.), it may be challenging to implement for narrow vessels due to potential obstruction of blood flow and a complicated deployment procedure. Thus, in some variations, the transducer(s) may be positioned on or near a vessel wall. In such variations, due to the application's constraints, the signal waveform transmitted and/or received by a WM's transducer may be at a non-zero angle, θ, to the direction of flow. Such a measurement may be called an ‘off-angle Doppler measurement’ or ‘off-angle Doppler estimation of fluid velocity.’ The challenge with such a measurement is that the Doppler shift is proportional to νcos θ, but θ may be large and/or not precisely known and, unlike external imagers, implantable transducers may not be amenable to dynamic re-positioning, thus, making the estimation of ν challenging. Solutions presented herein may be useful to overcome this challenge with off-angle Doppler measurements.

i. Using a Plurality of Transducers and Geometric Symmetry

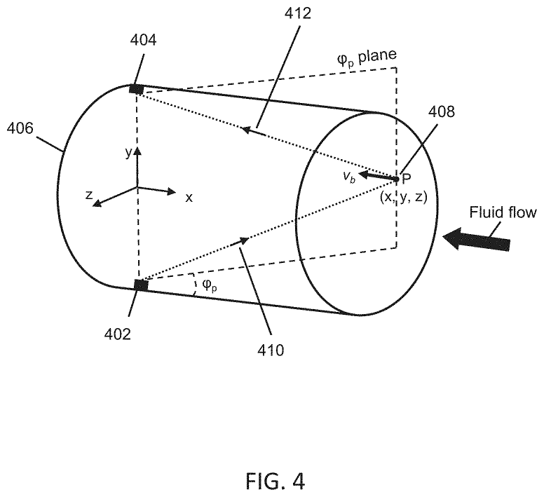

In some variations, a plurality of transducers and geometric symmetry may be used to reduce the angular dependence, allowing an accurate estimation of velocity, as explained in detail herein. One or more transducers may be positioned on or within a vessel (e.g., attached to a vessel wall). Such transducers may belong to one or more WMs, and may be located on an implantable device structure such as a stent, a prosthetic valve, and the like. As an example, FIG. 4 shows an ultrasonic transducer ‘s1’ (402) and an ultrasonic transducer ‘s2’ (404) positioned on a vessel wall (406). For example, the transducers may be positioned approximately diametrically opposite to each other. Transducer ‘s1’ (402) may be configured as a transmit transducer to transmit a transmitted signal waveform (410), and transducer ‘s2’ (404) may be configured as a receive transducer to receive a reflected signal waveform (412) generated at one or more reflection locations upon reflection of the transmitted signal waveform at least in part by the flowing fluid. Blood velocity at reflection location ‘P’ (408) with coordinates (x, y, z) in the vessel may be denoted by ν_{b}. The reflected signal waveform may have a frequency shift and/or a phase shift relative to the transmitted signal waveform due to Doppler effect. A processor of the WM may be configured to process the received signal waveform to generate waveform parameter data (e.g., a Doppler shift, frequency shift, phase shift, time delay, and the like), and estimate a physiological parameter of the patient (e.g., blood velocity, flow, and the like) from it.

Signal roundtrip time may be defined as the time elapsed between transmission of the transmitted signal waveform by the transmit transducer and reception of the reflected signal waveform by the receive transducer. The transmit and receive transducers may be time-synchronized such that the signal roundtrip time is known for given reflected signal waveforms. Since a given reflection location corresponds to a particular signal roundtrip time, signal roundtrip time may be used for range gating or setting the location(s), range or sample volume(s) at which velocity is to be measured. For a target reflection location, signal roundtrip time may be estimated based on the propagation speed of sound in the medium. Several variations of time-synchronizing the transmit and receive transducers are possible. In some variations, the transmit and receive transducers may be a part of the same WM, i.e., they may be electrically connected to a processor of the WM (e.g., as shown in FIG. 2), allowing the use of typical logic and timing circuits for time-synchronization and range gating. In some variations, the transmit and receive transducers may belong to physically separate WMs. In some variations, time-synchronization may be accomplished for physically separate WMs by transmitting a wireless synchronization signal between the two WMs and/or from the external wireless device to one or more WMs. Such a synchronization signal may be a radiofrequency (RF) signal, an infrared (IR) signal, an ultrasound signal, and the like, and may be in the form of a pulse or a set of pulses, and the like.

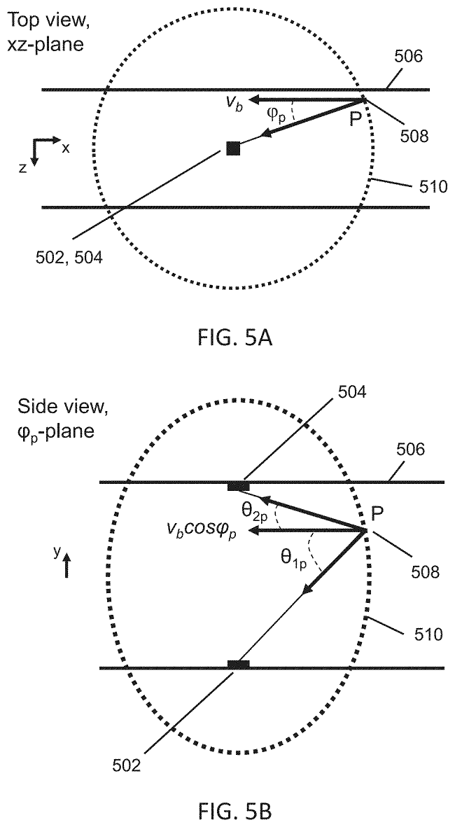

FIG. 5A and FIG. 5B show the top view and side view, respectively, of the geometry illustrated in FIG. 4. Denote the frequency of the transmitted signal waveform by ƒ₀ and speed of sound in blood by c. Angles ϕₚ, θ₁ₚ and θ₂ₚ are as shown in FIG. 5A and FIG. 5B. In some variations, the signal waveform may be an ultrasonic signal waveform (e.g., comprising one or more pulses), and ƒ₀ may be between about 0.1 MHz and about 100 MHz.

Consider first the case if only transducer ‘s1’ (502) was used (no ‘s2’), wherein ‘s1’ (502) is configured both as a transmit transducer and a receive transducer. Such a measurement may be referred to as a pulse-echo measurement. In this case, the component of blood velocity towards ‘s1’ (502) is given by ν_{b} cos ϕₚ cos θ₁ₚ. Based on Doppler effect, the frequency shift (Δƒ₁₁) of the reflected signal waveform (received by ‘s1’) relative to the transmitted signal waveform (transmitted by ‘s1’), may be derived as:

$$Δ {f}_{11} \approx ( \frac{2 {f}_{0} {v}_{b}}{c} ) \times cos {ϕ}_{p} cos {θ}_{1 p} \qquad ( 1 )$$

Since blood velocity inside the vessel is typically much larger than the velocity of any surrounding tissues, the Doppler shift in the received reflected signal waveform will correspond largely to blood velocity inside the vessel. For a given signal roundtrip time or given distance (R) from ‘s1’ at which velocity measurements are desired, ‘s1’ may simultaneously receive reflections from the surface of a sphere with radius R. Doppler shift in the received signal waveform, thus, largely corresponds to reflection locations on this sphere's surface intercepted by the vessel. For a single transducer ‘s1’, angles ϕₚ and θ₁ₚ may vary significantly over these reflection locations, leading to a large variation in Δƒ₁₁, making the estimation of ν_{b} challenging. Note that this result is also applicable if two separate transducers were used for transmit and receive, but positioned near each other on the same side of the vessel.

Now consider the case where ‘s1’ (502) is configured as a transmit transducer and ‘s2’ (504) is configured as a receive transducer (may be referred to as a pitch-catch measurement), wherein ‘s1’ (502) and ‘s2’ (504) may be located approximately opposite (e.g., diametrically opposite) to each other as illustrated in FIG. 4 and FIG. 5B. The component of blood velocity in the plane defined by ‘s1’ (502), s2′ (504) and ‘P’ (508) may be derived to be ν_{b} cos ϕₚ. Further, the component of blood velocity towards ‘s1’ (502) is ν_{b} cos ϕₚ cos θ₁ₚ and that towards ‘s2’ (504) is ν_{b} cos ϕₚ cos θ₂ₚ. Thus, based on Doppler effect, the total frequency shift (Δƒ₁₂) of the reflected signal waveform as received by ‘s2’ (504), relative to the transmitted signal waveform as transmitted by ‘s1’ (502), may be derived as:

$$Δ {f}_{12} \approx ( \frac{2 {f}_{0} {v}_{b}}{c} ) \times cos {ϕ}_{p} \times ( \frac{cos {θ}_{1 p} + cos {θ}_{2 p}}{2} ) = ( \frac{2 {f}_{0} {v}_{b}}{c} ) \times K \qquad ( 2 )$$

Here, ‘K’ represents the factors that depend on the angles. In this case, for a given signal roundtrip time, reflection locations corresponding to blood flow may lie on the surface of an ellipsoid (510) intercepted by the vessel (506), as shown in FIG. 5B. Due to geometric symmetry of the two transducers, for different reflection locations, if θ₁ₚ increases, then θ₂ₚ may decrease, and vice versa, leading to a reduced variability of Δƒ₁₂ over the reflection locations. In some variations, an approximate value of ‘K’ (e.g., 0.9 or 0.8 depending on the geometry of the vessel) may be used in equation (2) to estimate ν_{b} from Δƒ₁₂. As a result, estimation of blood velocity, and/or changes in blood velocity or flow (e.g., change over time), may be more accurate and reliable compared to the case when geometric symmetry is not leveraged for off-angle Doppler measurements. Note that this may be advantageous for any type of flow through the vessel, such as, plug, laminar, and the like.

Referring to FIG. 4, in some variations, a pitch-catch measurement may be done with s2′ (404) configured as the transmit transducer and ‘s1’ (402) configured as the receive transducer. In some variations, a two-way pitch-catch measurement may be performed between ‘s1’ (402) and ‘s2’ (404), i.e., ‘s1’ (402) transmits and ‘s2’ (404) receives, and vice versa, and the resulting Doppler shifts may be combined (e.g., added or averaged) to estimate blood velocity. In some variations, pulse-echo measurements may be performed using both ‘s1’ (402) and ‘s2’ (404) independently, and the resulting Doppler shifts may be combined (e.g., added or averaged) to estimate blood velocity. In some variations, two pairs of transducer elements may be used, one pair at the location of ‘s1’ (402), and another pair at the location of ‘s2’ (404), wherein one transducer element of a pair may be configured to transmit and the other transducer element of the pair may be configured to receive signal waveforms. Thus, in variations described herein, the use of a plurality of transducers positioned on a vessel wall, and geometric symmetry, may allow reduction in the angular dependence for off-angle Doppler estimation of fluid velocity.

ii. Using One or More Arrays of Transducers

In some applications, it may be important to measure fluid velocity in a specific region or reflection location within a vessel (e.g., near the center of a vessel's lumen). To achieve this, in some variations, one or more wireless monitors may comprise one or more ultrasonic arrays, comprising a plurality of ultrasonic transducer elements, and may be positioned on a vessel wall, wherein the one or more arrays may be used to beamform or focus the transmitted and/or received/reflected signal waveforms at the desired reflection location(s) by adjusting the phase or time delay between transducer elements. By beamforming or beam steering to a small focal spot at the desired reflection location (e.g., at a known angle), variation of the angle between blood velocity and the direction of propagation of the signal waveforms over the reflection locations may be reduced. Thus, the variation of measured Doppler shifts may be reduced, allowing an accurate estimation of blood velocity at the desired location. For instance, this angle may vary only by about ±5° over the focal spot, resulting in a relatively accurate estimation of blood velocity using the Doppler effect, compared to the case when no focusing or beamforming is done.

iii. Tilting of Transducers

In some applications, the beam width of a transducer may be limited (i.e., practical transducers may not be omni-directional or approximately omni-directional) such that it may not be possible to efficiently transmit or receive energy at large angles (e.g., between about 60° and about 90°) relative to the axis of the transducer's main lobe. For instance, this may be a limitation for narrow vessels where the desired reflection location may be at a large distance from the transducer (compared to the vessel diameter) along the length of the vessel, requiring transmission/reception at extreme angles if the transducer's main lobe is oriented orthogonal to the vessel's length. To mitigate this challenge, in some variations, one or more transducers may be positioned on a wireless monitor such that the transducer may be tilted or oriented to point the main lobe of the transducers' radiation pattern towards, or approximately towards, the desired reflection location. For example, in some variations, one or more transducers may be oriented on a vessel wall such that the axis of the main lobe of the transducers' radiation pattern is approximately parallel to the length of the vessel. In this way, the angle between blood velocity and the direction of propagation of the transmitted and received signal waveforms (θ₁ₚ and θ₂ₚ) may be kept within the −3 dB beam width of the transducers, allowing efficient transmission and reception of signal waveforms.

In some variations, any of the Doppler measurement techniques described above may be used independently or in combination. Velocity may be measured upstream and/or downstream relative to one or more transducers' location(s) in a vessel by measuring the corresponding positive and/or negative Doppler shifts, respectively. These techniques may be applied to measure or estimate one or more of velocity across the vessel's cross-section, volume flow, 2D/3D velocity maps, maximum/minimum velocity, mean velocity, combinations thereof, and the like.

b. Pulse Arrival Time Measurement

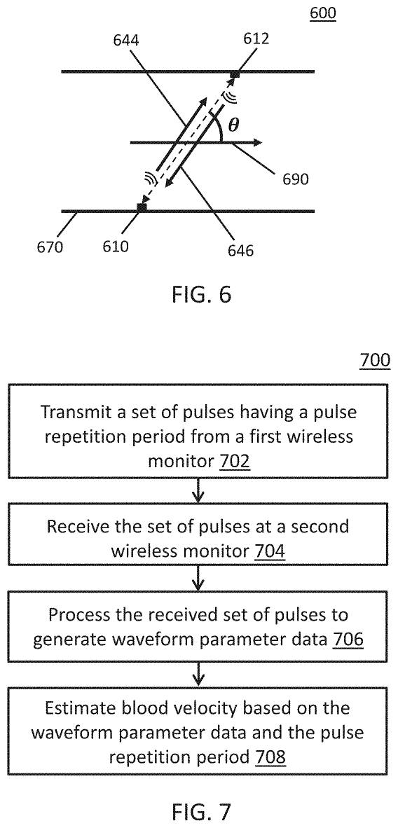

In some variations, a pulse arrival time measurement may be performed to estimate fluid velocity. FIG. 6 shows an illustrative view of a variation of a wireless monitoring system configured to measure fluid velocity. The wireless monitoring system (600) may comprise two wireless monitors (610, 612) positioned on or near walls of a vessel (670) containing fluid flow (690). Each wireless monitor (610, 612) may comprise one or more transducers (not shown) for receiving and/or transmitting signal waveforms. As shown before in FIG. 2, in some variations, transducers for receiving and transmitting signal waveforms may belong to the same wireless monitor. In some variations, the fluid may be blood, and the vessel may be a blood vessel or any cardiac chamber or region through which blood may flow. The wireless monitors (610, 612) may be configured to transmit and/or receive signal waveforms for blood velocity estimation via an uplink signal (644) and/or a downlink signal (646). In some variations, the wireless monitors (610, 612), or transducers thereof, may be positioned such that blood velocity at the desired measurement location(s) has a non-zero component along a propagation direction of the signal waveform, which may approximately be along the line joining the two transducers of the two wireless monitors (610, 612). For example, for the variation shown in FIG. 6, the component of blood velocity (ν) along the line joining the two wireless monitors (610, 612) is νcos θ. In some variations, in order to estimate blood velocity from the received signal waveform and the corresponding waveform parameter data, it may be useful to have θ not be equal to 90°, so that νcos θ is non-zero.

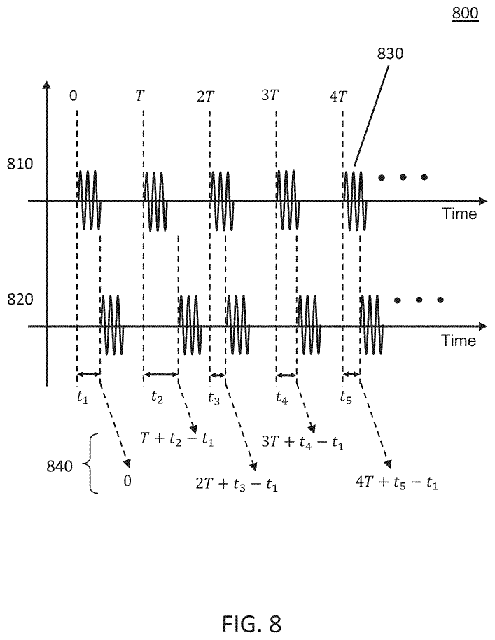

In some variations, the signal waveform may comprise a set of pulses with a pulse repetition period, and the waveform parameter data may comprise a set of pulse arrival times of the signal waveform. FIG. 7 is a flowchart that generally describes a variation of a method of estimating blood velocity using pulse arrival time measurement. In some variations, the external wireless device may transmit one or more downlink signals such as power and/or commands to one or more wireless monitors during and/or before the beginning of the measurement process (700). The measurement process (700) may begin with a first wireless monitor transmitting a signal waveform comprising a set of pulses having a pulse repetition period, T (702). In some variations, the pulses may be ultrasonic pulses with a sinusoidal waveform, a carrier frequency of between about 0.1 MHz and about 100 MHz and a pulse duration of between about 2 cycles to about 1000 cycles.

In some variations, T may be greater than about 100 μs. The value of T corresponds to the rate at which the velocity waveform is sampled. For example, T of about 1 ms may correspond to a sampling rate of about 1 kHz. In some variations, the value of T may be fixed (e.g., 1 ms) for a given measurement process (700). In some variations, the value of T may vary (e.g., smaller T, or a higher sampling rate, when velocity is varying at a faster rate) during a given measurement process (700). In some variations, the value of T (fixed or variable) may be predetermined and known a priori (e.g., hardcoded in memory) to one or more of the wireless monitors and the external wireless device. In some variations, the value of T may be communicated (e.g., using digital communication) by the first wireless monitor to one or more of the second wireless monitor and the external wireless device via an uplink signal. In some variations, the external wireless device may communicate the value of T to one or more wireless monitors via a downlink signal. In some variations, the value of T may be computed by the second wireless monitor and/or by the external wireless device by processing the received or measured signal waveform. For example, for wireless monitors located in the LVOT, during the period of a cardiac cycle when blood velocity is about zero (or constant), consecutive pulse arrival times at the second wireless monitor may be separated by a constant duration approximately equal to the pulse repetition period, T, and the second wireless monitor may process all pulse arrival times during a cardiac cycle to determine T.

The second wireless monitor may receive or measure the signal waveform comprising the set of pulses transmitted by the first wireless monitor (704). The received set of pulses may be processed to generate waveform parameter data (706). Waveform parameter data may comprise one or more of a set of pulse arrival times of the signal waveform and a set of differences in pulse transit times of consecutive pulses, as described in more detail herein. In some variations, the second wireless monitor may comprise a processor which may process the received set of pulses to generate waveform parameter data. Further, blood velocity may be estimated based on the waveform parameter data and the pulse repetition period (708), as described in more detail herein. In some variations, the processor of the second wireless monitor may estimate blood velocity based on the waveform parameter data and the pulse repetition period. Subsequently, the second wireless monitor may transmit the blood velocity data, and/or any parameter data derived from blood velocity, to the external wireless device via an uplink signal. In some variations, the second wireless monitor may transmit waveform parameter data, and/or the pulse repetition period, to the external wireless device via an uplink signal, and an external wireless device may estimate blood velocity, and/or any parameter derived from blood velocity, based on the waveform parameter data and the pulse repetition period.

FIG. 8 shows an example of a timing diagram (800) used for the estimation of blood velocity in this method. The first wireless monitor may transmit a signal waveform comprising a set of pulses (810) and the second wireless monitor may receive or measure a corresponding set of pulses (820). Each pulse (830) transmitted by the first wireless monitor may be a sinusoidal ultrasonic pulse as described before. It may be assumed that the pulses are transmitted by the first wireless monitor at times 0, T, 2T, 3T, and so on, as shown in FIG. 8. The transit or propagation times of pulses from the first wireless monitor to the second wireless monitor are denoted by t₁, t₂, t₃, and so on. The transit time of a pulse is dependent on the value of blood velocity at the time of propagation of the pulse. The set of pulse arrival times (840) as measured by the second wireless monitor, relative to the arrival time of the first pulse, are given by 0, (T+t₂−t₁), (2T+t₃−t₁), (3T+t₄−t₁), and so on, as shown in FIG. 8. In general, the arrival time of the n^{th} pulse is given by ((n−1)T+tₙ−t₁).

Using the value of T, the second wireless monitor and/or the external wireless device may further compute a set of differences in pulse transit times of consecutive pulses. The difference in pulse transit times of the n^{th} pulse and the (n+1)^{th} pulse may be denoted by Δtₙ, and is given by (tₙ₊₁−tₙ). The relationship between Δtₙ and other system parameters is given by equation (3):

$$Δ {t}_{n} = {t}_{n + 1} - {t}_{n} = \frac{L}{c - {v}_{n + 1} cos θ} - \frac{L}{c - {v}_{n} cos θ} \qquad ( 3 )$$

In equation (3), L is a propagation distance of the signal waveform between the first wireless monitor and the second wireless monitor, c is the speed of sound in blood, and θ is the angle between a longitudinal axis of the signal waveform, or the line joining the first and second wireless monitors, and the propagation direction of the blood. νₙ₊₁ and νₙ are the (n+1)^{th} and n^{th} samples of blood velocity, respectively. Here, it is assumed that the second wireless monitor which receives the set of pulses is upstream from the first wireless monitor that transmits the set of pulses, i.e., pulses propagate opposite to the direction of blood flow. Due to this, equation (3) has a “minus” sign between c and νₙ₊₁ cos θ or νₙ cos θ. If the receiver of the set of pulses is downstream from the transmitter, i.e., if pulses propagate along the direction of blood flow, then equation (3) will have a “plus” sign between c and νₙ₊₁ cos θ or νₙ cos θ. Equation (3) may be further simplified as:

$$Δ {t}_{n} = {t}_{n + 1} - {t}_{n} \approx \frac{L cos θ}{{c}^{2}} ( {v}_{n + 1} - {v}_{n} ) = \frac{L cos θ}{{c}^{2}} Δ {v}_{n} \qquad ( 4 )$$

Here, the difference between consecutive blood velocity samples, νₙ₊₁ and νₙ, is denoted by Δνₙ. Equation (4) may be further re-arranged to give:

$$Δ {v}_{n} \approx \frac{{c}^{2} Δ {t}_{n}}{L cos θ} \qquad ( 5 )$$

By starting from the point where blood velocity is approximately zero or constant, changes in blood velocity between consecutive samples, as given by equation (5), may allow estimation of blood velocity as a function of time. Blood velocity may be estimated over one or many cardiac cycles in this way. In some variations, a two-way transmission of a set of pulses between the two wireless monitors may be performed. For example, this may be done in order to reduce the sensitivity of blood velocity calculation to parameters like L or θ. In some variations, other mathematical simplifications may be performed, starting from equation (3), for the estimation of blood velocity.

In some variations, pulse transit times, or a difference in pulse transit times between consecutive pulses, Δtₙ, may be useful to measure a change in L. For example, for two wireless monitors positioned on a stent, the distance between the two wireless monitors may change during a cardiac cycle due to motion of the blood vessel walls. In some variations, equation (3) may be adapted to represent Δtₙ as a function of Lₙ₋₁ and Lₙ, which may be the corresponding distances between the first and second wireless monitors at the time of transmission of the (n+1)^{th} and the n^{th} pulses, respectively. In some variations, blood velocity may be assumed to be a constant. Thus, in such variations, based on equation (3), Δtₙ may be directly proportional to (Lₙ₊₁−Lₙ), since the denominator of the two terms in equation (3), given by (c−νcos θ), may be common, where ν may denote a constant, or a relatively constant, blood velocity during this measurement process. In some variations, a third wireless monitor may be positioned such that the line joining the first and the third wireless monitors is perpendicular, or approximately perpendicular, to blood velocity (i.e., 0 may be equal to, or approximately equal to, 90°). The third wireless monitor may also receive the signal waveform transmitted by the first wireless monitor. In such variations, an equation for Δtₙᵢ, similar to equation (3), may be written for the signal waveform received by the third wireless monitor, where cos θ may be zero or very small. Thus, in such variations, Δtₙ may be more sensitive to changes in the distance between the two wireless monitors compared to changes in blood velocity. In some variations, after estimating a change in the distance between the first and the third wireless monitors, a change in the distance between the first and the second wireless monitors may be estimated based on the geometry, and this may be used to more accurately estimate blood velocity from equation (3). Such a technique of estimating the distance between two wireless monitors, or a change in the distance between two wireless monitors, may be useful to accurately estimate blood velocity, determine one or more of a diameter or cross-section of a blood vessel, a dimension of a cardiac structure (e.g., LVOT diameter, valve or prosthetic valve diameter), changes in a dimension as a function of time (e.g., over one or more cardiac cycle, over long-term), any combinations or derivates thereof (e.g., cross-sectional area), and the like.

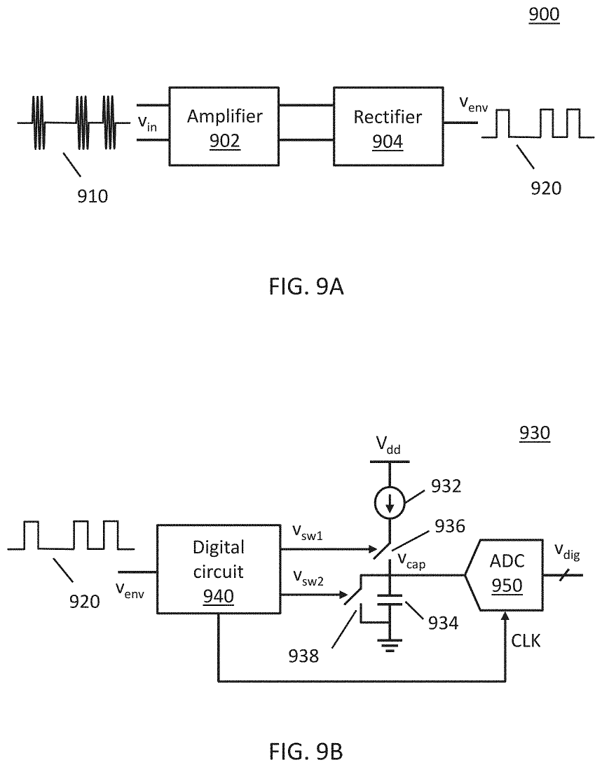

In some variations, the processor of the second wireless monitor may comprise a pulse detector for measuring the signal waveform that comprises a set of pulses. In some variations, the pulse detector may comprise one or more of an envelope detection circuit (e.g., a rectifier, a low pass filter), energy detection circuit, amplifier, comparator, combinations thereof, and the like. FIG. 9A is an illustrative block diagram of a pulse detector implemented in the form of an envelope detection circuit (900). The envelope detection circuit may comprise an amplifier (902) connected to a rectifier (904). The input of the amplifier, yin, may be connected to a transducer (not shown) that receives a signal waveform comprising a set of pulses (910). An envelope signal (920) of the signal waveform may be generated at the output of the rectifier, vout. This envelope signal (920) may be used for pulse arrival time detection, as described in more detail herein.

In some variations, the processor of the second wireless monitor may further comprise a pulse arrival time detector for measuring a set of pulse arrival times of the signal waveform from its envelope signal. In some variations, the pulse arrival time detector may comprise one or more of a time-to-digital converter (TDC) circuit, an integrator circuit, a sampling circuit, an analog-to-digital converter (ADC) circuit, a timer circuit, a clock, a counter, an oscillator, a phase-locked loop (PLL), a frequency locked loop (FLL), combinations thereof, and the like. FIG. 9B is an illustrative block diagram of a pulse arrival time detector (930). The pulse arrival time detector (930) may comprise a current source (932) that may charge a capacitor (934) depending on the state of a first switch (936) and a second switch (938), as shown in FIG. 9B. The first switch (936) and the second switch (938) may be controlled via switch control signals ν_{sw1} and ν_{sw2}, respectively, that are generated by a digital circuit (940) which may have the envelope signal (920) as its input. For example, upon the detection of a rising edge of the envelope signal (920), the first switch (936) may be turned off and the second switch (938) may be turned on for a short duration to fully discharge capacitor (934) to zero voltage. Immediately after this short duration, the first switch (936) may be turned on and the second switch (938) may be turned off until the detection of the next rising edge of the envelope signal (920), in order to charge capacitor (934) from zero voltage to a voltage, ν_{cap}, that is proportional to the time duration between the two consecutive rising edges of the envelope signal (920). The voltage ν_{cap} may be sampled and digitized by an analog-to-digital converter or ADC (950) to generate a digital code, ν_{dig}, at its output. A sampling clock (CLK) for the ADC (950) may be generated by the digital circuit (940). The digital code, ν_{dig}, generated by the ADC may be representative of the time duration between two consecutive rising edges of the envelope signal (920), which corresponds to the difference between the arrival times of two consecutive pulses at the second wireless monitor. For example, for the n^{th} and the (n+1)^{th} pulses received by the second wireless monitor, this digital code corresponds to (T+tₙ₊₁−tₙ), where T is the pulse repetition period, and tₙ and tₙ₊₁ are the transit or propagation times of the n^{th} and the (n+1)^{th} pulses, respectively, from the first wireless monitor to the second wireless monitor. As an example, in some variations, blood velocity may be sampled at about 100 Hz over about 10 cardiac cycles, the ultrasonic pulse repetition period, T, may be about 10 ms, pulse carrier frequency may be about 1 MHz, and the duration of each pulse may be about 20 μs.

c. Phase Measurement

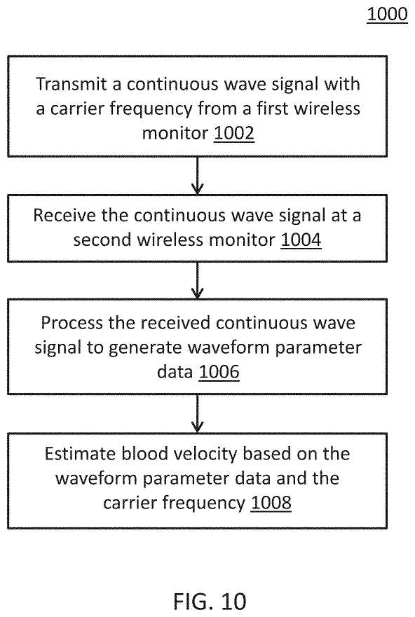

In some variations, the signal waveform may comprise a continuous wave (CW) signal with a carrier frequency, and the waveform parameter data may comprise a set of phase shifts of the signal waveform relative to one or more reference phases of the signal waveform. In some variations, a configuration of two wireless monitors, as shown in FIG. 6, may be used. FIG. 10 is a flowchart that generally describes a variation of a method of estimating blood velocity using phase measurement. Several variations and steps discussed for pulse arrival time measurement above may also be applicable herein. The measurement process (1000) may begin with a first wireless monitor transmitting a signal waveform comprising a CW signal with a carrier frequency (1002). In some variations, the CW signal may be an ultrasonic CW signal with the carrier frequency of between about 0.1 MHz and about 100 MHz.

In some variations, one or more of the carrier frequency and one or more reference phases of the CW signal may be predetermined and known a priori (e.g., hardcoded in memory) to one or more of the wireless monitors and the external wireless device, may be communicated by the first wireless monitor to one or more of the second wireless monitor and the external wireless device via an uplink signal, may be communicated by the external wireless device to one or more wireless monitors via a downlink signal, and/or may be computed by the second wireless monitor and/or by the external wireless device by processing the received or measured signal waveform. For example, for wireless monitors located in the LVOT, during the period of a cardiac cycle when blood velocity is about zero, the phase of the received signal waveform may be designated as a reference phase, and may be normalized to zero, by the second wireless monitor.

The second wireless monitor may receive or measure the signal waveform comprising the CW signal transmitted by the first wireless monitor (1004). The received CW signal may be processed to generate waveform parameter data (1006). Waveform parameter data may comprise a set of phase shifts of the signal waveform, relative to one or more reference phases of the signal waveform. Further, blood velocity may be estimated based on the waveform parameter data and the carrier frequency (1008). In some variations, the processor of the second wireless monitor may estimate blood velocity based on the waveform parameter data and the carrier frequency. Subsequently, the second wireless monitor may transmit the blood velocity data, and/or any parameter data derived from blood velocity, to the external wireless device via an uplink signal. In some variations, the second wireless monitor may transmit waveform parameter data, and/or the carrier frequency, to the external wireless device via an uplink signal, and an external wireless device may estimate blood velocity, and/or any parameter derived from blood velocity, based on the waveform parameter data and the carrier frequency.

In some variations, a first wireless monitor may transmit a signal waveform comprising a CW signal, given by A₀ sin(2πƒ₀t). Here, ƒ₀ is the carrier frequency and A₀ is the amplitude. In some variations, the carrier frequency ƒ₀ for an ultrasound CW signal may be between about 0.1 MHz and about 100 MHz. The second cardiac monitor may receive or measure a signal waveform, given by Aₗ sin(2πƒ₀t+Δϕ(t). Here, the phase shift Δϕ(t) is proportional to the net component of blood velocity along the propagation direction of the signal waveform. In variations where the wireless monitoring system may be disposed in an LVOT, blood velocity in the LVOT may be approximately zero before the opening of an aortic valve. During this time, the phase of the signal received by the second wireless monitor may be approximately constant, which may be considered as a reference phase. This constant reference phase may be normalized to zero. Thus, Δϕ(t)=0 when ν(t)=0. Blood velocity at time t (i.e., ν(t)), may then be estimated from Δϕ(t) since blood velocity may be proportional to phase shift relative to a reference phase. Blood velocity may be estimated over one or many cardiac cycles.

For example, if ƒ₀ is 1 MHz, then each cycle of the received signal waveform has a duration of 1 μs. In some variations, the processor of the second wireless monitor may comprise a phase detector that may process the received signal waveform in a time window of 1 ms to calculate an average phase shift of all the cycles in that time window. This phase shift may be related to an average velocity value during the 1 ms duration. In this manner, the phase detector may process the received signal waveform in consecutive 1 ms time windows, resulting in a 1 kHz sampling rate of blood velocity. Using the phase measurement method, blood velocity may be sampled at any desired sampling rate such as 1 kHz, 10 kHz, 100 Hz, and the like. In some variations, the phase of the received signal waveform may be processed in other ways to estimate blood velocity.

Given that L is a propagation distance of the signal waveform between the first wireless monitor and the second wireless monitor, c is the speed of sound in blood, and θ is the angle between a longitudinal axis of the signal waveform, or the line joining the first and second wireless monitors, and the propagation direction of the blood, time shift Δt is given by equation (6):

$$Δ t = \frac{L}{c - v cos θ} - \frac{L}{c} \qquad ( 6 )$$

In the derivation of equation (6), it is assumed that the second wireless monitor which receives the CW signal is upstream from the first wireless monitor that transmits the CW signal, i.e., the signal waveform propagates opposite to the direction of blood flow. Due to this, equation (6) has a “minus” sign between c and νcos θ. If the receiver of the CW signal is downstream from the transmitter, i.e., if the signal waveform propagates along the direction of blood flow, then equation (6) will have a “plus” sign between c and νcos θ, and Δt will accordingly have a negative value. Phase shift Δϕ is related to the time shift Δt by the carrier frequency ƒ₀, and may be given by:

$$Δ ϕ = 2 π {f}_{0} \times Δ t \approx \frac{2 π {f}_{0} Lv cos θ}{{c}^{2}} \qquad ( 7 )$$

The final approximation in equation (7) for Δϕ is based on νcos θ»c, which may be reasonable since maximum blood velocities may be less than about 1% of the speed of sound in blood. Solving for blood velocity may give:

$$v ( t ) \approx \frac{{c}^{2}}{2 π {f}_{0} L cos θ} \times Δ ϕ ( t ) \qquad ( 8 )$$

In some variations, other types of mathematical simplifications may be performed for the estimation of blood velocity. In some variations, no mathematical simplifications may be made, and equation (6) may be used to solve for blood velocity in terms of the time shift (which may be written in terms of the phase shift). In some variations, equation (6) may be adapted and written in terms of a change in L. As discussed for the pulse arrival time measurement technique, in variations where ν may be assumed to be approximately constant or about zero, and/or θmay be about 90°, a change in L may be estimated based on Δt or Δϕ.

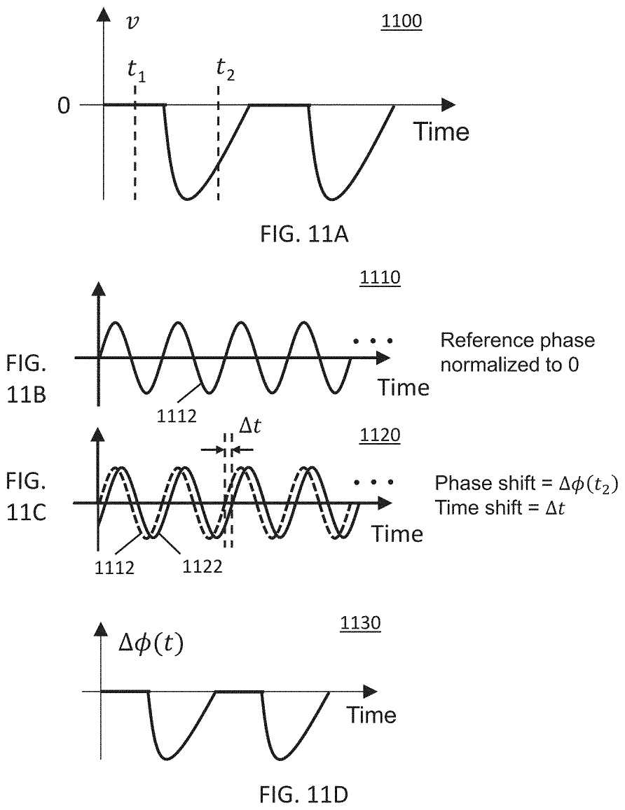

FIGS. 11A-11D are illustrative plots showing the relationship between phase shift of a received signal waveform and blood velocity. FIG. 11A is an illustrative plot (1100) of an example blood velocity waveform in the LVOT over time. FIG. 11B is an illustrative plot (1110) of the received CW signal (1112) over time beginning at time t₁ (shown in FIG. 11A) where the phase of the received signal waveform is normalized to zero, corresponding to zero blood velocity. FIG. 11C is an illustrative plot (1120) of the received CW signal (1122) over time beginning at time t₂ (shown in FIG. 11A) overlaid with the CW signal (1112) of FIG. 11B extrapolated to time t₂. As shown in FIG. 11C, CW signal (1122) has a time shift Δt, and a corresponding phase-shift Δϕ(t₂), compared to the CW signal (1112), due to non-zero blood velocity at time t₂. In general, the phase shift at any time t, denoted by Δϕ(t), may be obtained by considering the received CW signal in a small time window near time t (e.g., a 100 μs time window following time t) and comparing it to the phase of CW signal (1112) extrapolated to time t. FIG. 11D is an illustrative plot (1130) of the phase shift Δϕ(t), or a set of phase shifts, as a function of time.

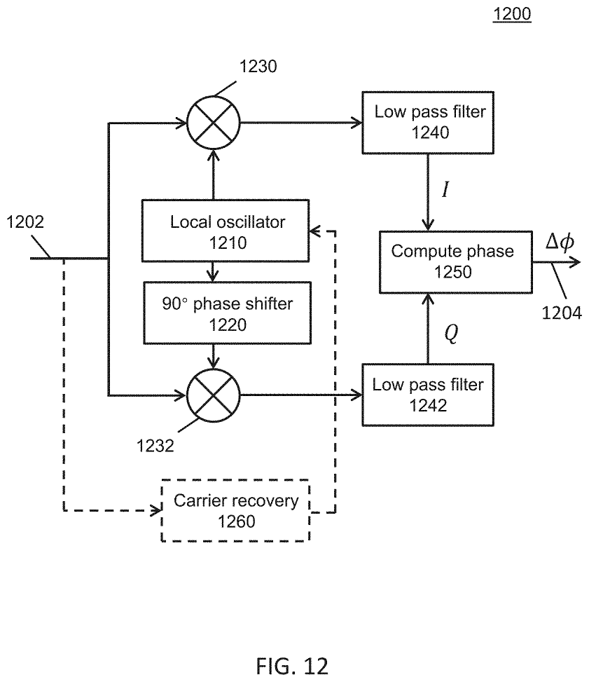

In some variations, the processor of the second wireless monitor may comprise a phase detector for measuring a set of phase shifts of the received signal waveform. In some variations, the phase detector may be configured to determine the carrier waveform based on the measured signal waveform and perform coherent phase demodulation by comparing the phase of the measured signal waveform with the phase of the determined carrier waveform. In some variations, the phase detector may be configured to perform non-coherent phase demodulation. FIG. 12 is an illustrative block diagram of a phase detector (1200) configured to generate a set of phase shifts. The input (1202) to the phase detector may be the received CW signal (1122) and its output (1204) may be a corresponding phase shift (Δϕ) The phase detector (1200) may comprise an IQ demodulator circuit comprising a local oscillator (1210), a 90° phase shifter (1220), mixers (1230, 1232), low pass filters (1240, 1242), and a block to compute phase (1250) from the I and Q signals, as shown. The received CW signal (1122) may be mixed with a signal generated by the local oscillator (1210) and the 90° phase shifter (1220), which may have a reference phase (e.g., zero phase), and low pass filtered in order to generate I and Q signals, and subsequently compute a phase shift (Δϕ) of the received CW signal relative to this reference phase. In some variations, a block for carrier recovery (1260) may be included to recover the original carrier signal, and/or a reference phase, from the input (1202), and provide this as an input to the local oscillator (1210). In some variations, data about the frequency and/or a reference phase of the carrier waveform may be transferred to the second wireless monitor by the first wireless monitor and/or the external wireless device via a downlink signal. In some variations, the processor of a second wireless monitor may compute a fast Fourier transform (FFT) of the received CW signal to compute a set of phase shifts.

d. Using a Plurality of Pressure Sensors to Estimate Velocity or Flow

In some variations, one or more wireless monitors comprising a plurality of pressure sensors may be used to estimate fluid velocity, flow and/or acceleration. The plurality of pressure sensors may belong to the same or different wireless monitors. One or more processors of the wireless monitor and/or an external wireless device may process the pressure (e.g., fluid pressure) measured by one or more wireless monitors to estimate a fluid velocity, flow and/or acceleration. In some variations, temporal profile of blood pressure (e.g., pressure waveforms), or pressure values at certain instances of time (e.g., at diastole, at systole, and the like), measured by the pressure sensors, may be processed to estimate blood velocity or flow near the pressure sensor locations or in the region between them. In some variations, Bernoulli's equation or principle may be used to estimate velocity or flow from pressure measurements. In some variations, a time shift or delay between pressure waveforms or peaks measured by two pressure sensors may be used to estimate blood velocity or flow. For example, pressure sensors may be positioned upstream and downstream of a heart valve or a blood vessel to estimate blood flow through the valve or vessel. As another example, a plurality of pressure sensors may be positioned along the circumference of a valve (e.g., attached to a prosthetic heart valve or stent), and relative differences in measured pressure values (e.g., during a ventricular diastole, or waveforms over one or more cardiac cycles) may be used to detect locations of paravalvular leaks and/or CE in the valve, and/or to create a spatial map of blood flow along the circumference of the valve. Such techniques of using a plurality of pressure sensors to estimate blood flow may be used to diagnose or monitor one or more conditions such as paravalvular regurgitation, aneurysms, restenosis, heart failure, prosthetic valve dysfunctions, and the like.

e. Local Density Measurement

In some variations, one or more wireless monitors may be configured to transmit a signal waveform into the bloodstream, and the same or a different wireless monitor may be configured to receive a corresponding reflected signal waveform. The received signal waveform may be processed to generate waveform parameter data comprising one or more of local density of blood, a related parameter such as number of one or more types of cells or contents in blood, and the like. For example, before the aortic valve opens, blood may collect temporarily in the LVOT. For a small duration immediately after the opening of the aortic valve, blood may experience acceleration into the aorta. During this time, blood plasma may flow out of the valve first due to its lower density, which may be about 1025 kg/m³, compared to the density of blood cells, which may be about 1125 kg/m³. As a result, temporarily there may be a higher local density of blood in the LVOT. As blood flows through the aortic valve, local blood density may eventually come back to the average blood density value of about 1060 kg/m³. Changes in local density of blood over time, and/or changes in the number of blood cells in a given region over time, may be used to estimate blood velocity, acceleration and/or flow. In some variations, the signal waveform to estimate local blood density or number of blood cells may comprise an electrical signal generated and/or measured using electrodes (e.g., to perform an electrical impedance measurement).

f. Transit-Time Measurements

In some variations, two or more transducers (e.g., ultrasonic transducers) belonging to one or more wireless monitors may be configured to perform signal transit-time measurements to estimate blood velocity. For example, two ultrasonic transducers may be positioned on/near a blood vessel wall such that blood velocity has a non-zero component along a line joining the two transducers (e.g., as was shown in FIG. 6). One-way or two-way transit-time measurements may be performed between the two transducers, wherein one transducer may be configured to transmit a signal waveform and the other transducer may be configured to receive the corresponding signal waveform. A processor of the wireless monitor may be configured to generate waveform parameter data comprising a transit time or propagation time of the signal waveform between the two transducers, which may then be used to estimate blood velocity based on the distance between the two transducers.

In order to accurately estimate the signal transit time between the two transducers, it may be important to know both the time at which a signal waveform was transmitted and the time at which it was received, i.e., time synchronization between the two transducers may be important. In some variations, the two transducers may belong to the same wireless monitor and, thus, may be electrically connected to a common processor, allowing the use of typical logic and timing circuits for time synchronization. In some variations, where the two transducers may belong to separate wireless monitors, time synchronization may be performed via an RF or an IR synchronization pulse transmitted by one wireless monitor to the other, or transmitted by the external wireless device to the WMs. Such an RF or IR pulse may travel instantaneously relative to ultrasound signal waveforms, and help with establishing a time reference for the WMs. In some variations, handshake between the external wireless device and one or more WMs may be performed using ultrasonic signals for time synchronization.

B. Physiological Structure Measurement

Generally, the methods described here may include estimation of a property of a physiological structure using a wireless monitoring system.

### a. Cardiac Structure Measurement Example

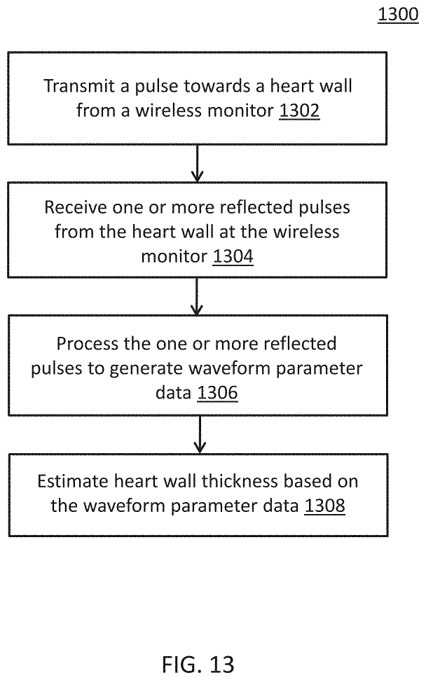

In some variations, a signal waveform may be transmitted by a wireless monitor toward a cardiac structure (e.g., a heart wall), and the physiological parameter of the patient may comprise a cardiac structure parameter (e.g., heart wall thickness). FIG. 13 is a flowchart that generally describes a variation of a method of estimating heart wall thickness using the wireless monitoring system described herein. The process (1300) may begin with a wireless monitor transmitting a pulse (e.g., an ultrasonic pulse) towards a heart wall (1302). In some variations, the wireless monitor may be located on an inner surface of a heart wall and may transmit a pulse towards an outer surface of the heart wall. When the pulse reaches the outer surface of the heart wall, a part of this pulse may undergo a reflection and the reflected pulse may travel back towards the wireless monitor. The wireless monitor may receive or measure one or more reflected pulses from the heart wall (1304). The one or more reflected pulses may be processed to generate waveform parameter data (1306). Waveform parameter data may comprise a time duration corresponding to the one or more reflected pulses, as described in more detail herein. In some variations, the wireless monitor may comprise a processor that may process the one or more reflected pulses to generate waveform parameter data. Further, heart wall thickness may be estimated based on the waveform parameter data (1308), as described in more detail herein. In some variations, the processor of the wireless monitor may estimate heart wall thickness based on the waveform parameter data. Subsequently, the wireless monitor may transmit the heart wall thickness data, and/or any parameter data derived from heart wall thickness, to the external wireless device via an uplink signal. In some variations, the wireless monitor may transmit waveform parameter data to the external wireless device via an uplink signal, and an external wireless device may estimate heart wall thickness, and/or any parameter derived from heart wall thickness, based on the waveform parameter data.

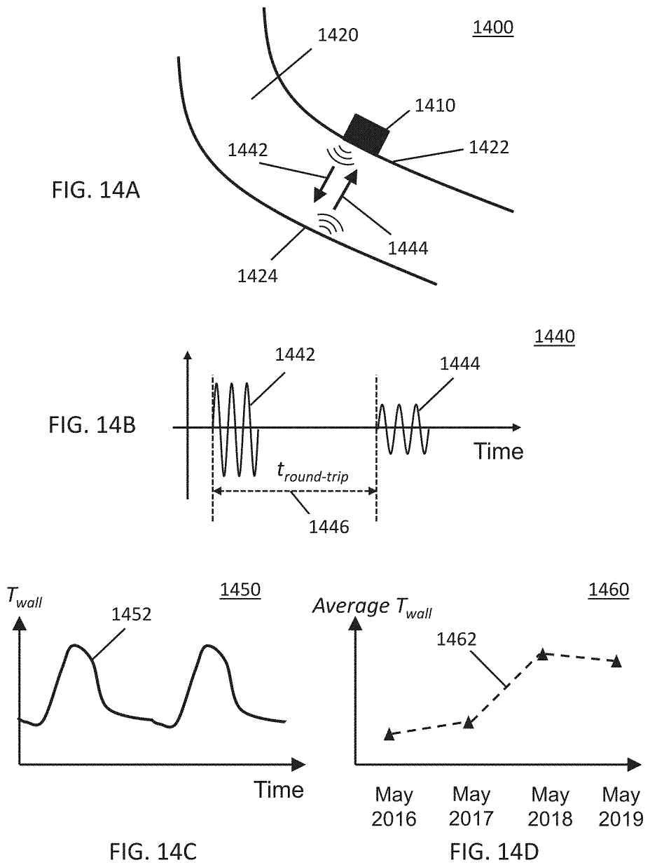

In some variations, a wireless monitoring system (1400) may be used for measurement of a heart wall thickness, as illustrated in FIG. 14A. A wireless monitor (1410) may be placed on an inner surface (1422) of a heart wall (1420). For example, a wireless monitor may be placed on the inner wall of the left ventricle. The wireless monitor may transmit a signal waveform (1442) towards the heart wall (1420). For example, the signal waveform may comprise an ultrasonic pulse. A reflected signal waveform (1444) may be generated at an outer surface (1424) of the heart wall (1420). The wireless monitor (1410) may measure the reflected signal waveform (1444). FIG. 14B shows an example timing diagram of the transmitted signal waveform (1442) comprising an ultrasonic pulse, and the reflected signal waveform (1444) comprising a reflected ultrasonic pulse which is received or measured by the wireless monitor (1410). As shown in FIG. 14B, the wireless monitor (1410) may measure a round trip time of the reflected ultrasonic pulse or, in other words, the time duration between the transmission of the signal waveform (1442) and reception of the reflected signal waveform (1444) by the wireless monitor (1410).

Based on the measured round trip time, t_{round-trip}, the heart wall thickness may be given by:

$${T}_{wall} = {c}_{wall} \times \frac{{t}_{round - trip}}{2} \qquad ( 9 )$$

In equation (9), T_{wall} is the heart wall thickness and c_{wall} is the speed of sound in the heart wall. A wireless monitor may calculate T_{wall} in this way at several time points during a cardiac cycle and T_{wall} may be plotted as a function of time over one or more cardiac cycles, as illustrated in FIG. 14C. Further, a metric related to T_{wall} over a cardiac cycle, such as an average value of T_{wall} over one cardiac cycle, or the value of T_{wall} at a specific point in the cardiac cycle (e.g., end-diastolic value) may be monitored over time. For example, FIG. 14D shows an illustration of an average value of T_{wall} measured over one cardiac cycle plotted over several years in order to monitor a long-term trend in heart wall thickness. This may be helpful for monitoring or assessing the progression of heart failure in a patient.

In some variations, a wireless monitor may have a part or a section that extends into, or is positioned inside, the heart wall. Such a part or section of the wireless monitor may comprise one or more pressure sensors to measure pressure inside the heart wall. In some variations, an entire wireless monitor may be embedded inside a heart wall and may measure pressure inside the heart wall. For example, a wireless monitor may have a part or section that extends into, or is positioned inside, the wall of an LV to measure pressure inside the wall or muscle of the LV. The wireless monitor may measure a waveform of pressure in the heart wall over one or more cardiac cycles. A parameter related to this pressure (e.g., an average value of pressure inside the heart wall over one or more cardiac cycles, pressure inside the heart wall at a particular point in the cardiac cycle) may be plotted over several years in order to monitor a long-term trend in the heart wall pressure. This may be helpful for monitoring or assessing one or more of the contraction or contractility of the heart wall, volume or mass of a heart chamber (e.g., LV), progression of heart failure of a patient, combinations thereof, and the like.

In some variations, one or more wireless monitors may be implanted in a heart chamber (e.g., LV) in order to measure a volume of the heart chamber. For example, two wireless monitors may be implanted inside the LV along an axis of the LV, and may perform a distance measurement between each other in order to measure an inner dimension of the LV. Distance measurements from one or more such pairs of wireless monitors may be incorporated to estimate a volume of the LV. Such measurements may be used to plot a waveform of volume of the heart chamber over one or more cardiac cycles. In some variations, wireless monitors may perform imaging of the heart chamber to measure distances between two points inside a heart chamber, and/or measure a volume of the heart chamber. In some variations, one or more wireless monitors implanted inside a heart chamber may be used as markers by an external wireless device to estimate a volume of a heart chamber. For example, an external wireless device may perform one or more of imaging of the wireless monitors, handshake with the wireless monitors, and the like, to determine their position or trace their path during one or more cardiac cycles. The external wireless device may estimate a volume of the heart chamber based on this measurement. In some variations, a wireless monitor may additionally measure blood pressure in a heart chamber. Such pressure and volume measurements in a heart chamber may be utilized to plot pressure-volume loops for the heart chamber.

In some variations, a wireless monitoring system as described herein may be used to monitor a heart valve and/or a prosthetic heart valve. For example, a wireless monitor positioned near a prosthetic valve leaflet may be configured to measure a signal waveform transmitted through one or more valve leaflets. In some variations, the signal waveform may be transmitted by the same wireless monitor (via the same or a different transducer) for reflection at the one or more leaflets (e.g., for use in imaging of the leaflet). In some variations, the signal waveform may be transmitted by a second wireless monitor (or a second transducer of the wireless monitor) positioned on the other side of the valve leaflet (for transmission of the signal waveform through one or more leaflets), or on the same side of the valve leaflet (for reflection of the signal waveform from one or more leaflets). In some variations, waveform parameter data may comprise one or more of an amplitude, a change in amplitude, a phase, a transit time or arrival time, a frequency, combinations thereof, and the like, of an ultrasonic signal waveform propagating through the one or more valve leaflets. Waveform parameter data may be indicative of the ultrasonic or mechanical properties of the leaflet(s). Such waveform parameter data may be processed and/or tracked over time (e.g., over one or more cardiac cycles, days, months, years) to estimate one or more of motion, thickness and deterioration (e.g., calcification) of the leaflet(s). For example, a reduction in the amplitude of the signal waveform over time may indicate calcification or leaflet deterioration.

b. Using One or More Pressure Sensors to Monitor a Physiological Structure

In some variations, a physiological structure such as one or more prosthetic valve leaflets may be monitored using one or more wireless monitors comprising one or more pressure sensors. One or more processors of the wireless monitor and/or an external wireless device may process the pressure measured by one or more wireless monitors to monitor a physiological structure or estimate a physiological parameter of a patient. For example, one or more pressure sensors may be positioned upstream and/or downstream from one more valve leaflets. For example, three pressure sensors may be deployed in the LVOT, each pressure sensor upstream from each leaflet of the aortic valve. Pressure values or temporal waveforms may be measured at these sensors and compared with each other to assess motion, thickness and/or deterioration/malfunction of one or more valve leaflets. For example, if a valve leaflet opens suddenly during a cardiac cycle, blood may start flowing at a high velocity near the corresponding pressure sensor. This may result in conversion of some of the pressure energy of blood near this pressure sensor into kinetic energy, resulting in a sudden drop in blood pressure measured by this sensor. Such a sudden drop in pressure may not result if a valve leaflet opens slowly or does not open fully due to leaflet deterioration (e.g., calcification, increased leaflet thickness, and the like). In some variations, pressure measurements from one or more pressure sensors may be processed to monitor one or more of a heart wall thickness, heart wall motion, heart wall contractility, size of a heart chamber (e.g., LV), and the like.

### C. Wireless Monitoring System Examples

The specific examples and descriptions herein are exemplary in nature and variations may be developed by those skilled in the art based on the material taught herein without departing from the scope of the present invention, which is limited only by the attached claims.

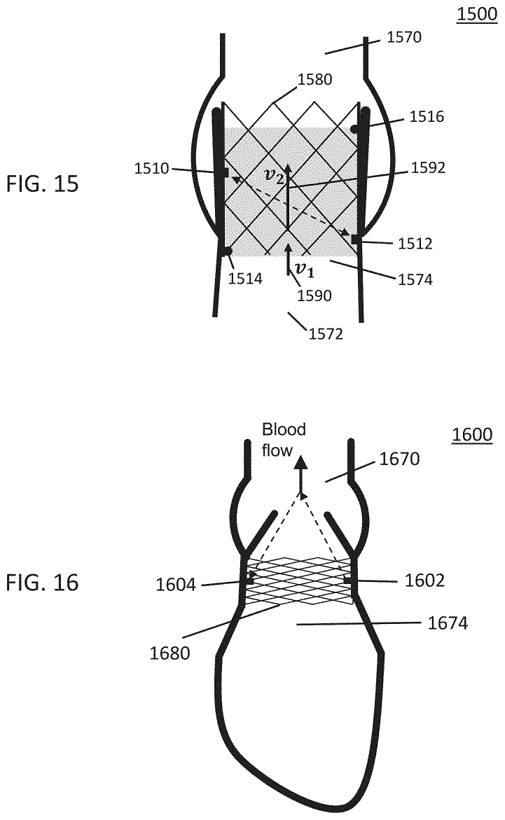

In some variations, as shown in FIG. 15, a wireless monitoring system (1500) may comprise a prosthetic aortic valve (1580) positioned between the aorta (1570) and the left ventricle (1572). A part of the prosthetic aortic valve (1580) may extend into the left ventricular outflow tract or LVOT (1574). A first wireless monitor (1510) and a second wireless monitor (1512) may be coupled (e.g., attached) to the prosthetic aortic valve (1580) for measuring blood velocity ν₂ (1592) at or near the aortic valve. These two wireless monitors may measure blood velocity using any technique taught herein. Further, a third wireless monitor (1514) may be attached to the prosthetic aortic valve (1580) in the LVOT (1574) and may measure blood pressure in the LVOT (1574), denoted by P₁. A fourth wireless monitor (1516) may be attached to the prosthetic aortic valve (1580) in or near the aorta (1570) and may measure blood pressure in or near the aorta (1570), denoted by P₂. These pressure measurements may be used to compute a pressure gradient, ΔP, given by:

ΔP=P₁−P₂ (10)

In some variations, the wireless monitors (1514, 1516) may transmit their pressure data to the external wireless device via an uplink signal, and the external wireless device may compute the pressure gradient. In some variations, a wireless monitor (1510, 1512) may transmit/receive a signal waveform and transmit waveform parameter data to the external wireless device, and the external wireless device may estimate blood velocity ν₂ (1592) based on waveform parameter data.

In some variations, the external wireless device may further estimate blood velocity in the LVOT, ν₁ (1590), based on the measured pressure gradient (ΔP) and estimated blood velocity at the aortic valve or in the aorta, ν₂ (1592) using the Bernoulli equation:

ΔP=P₁−P₂≈4(ν₂²−ν₁²) (11)

In some variations, after estimating ν₁, the external wireless device may further compute a velocity-time integral in the LVOT, denoted by VTI_{LVOT}, by integrating ν₁ over time. The external wireless device may further compute stroke volume (SV), given by:

SV=VTI_{LVOT}×CSA_{LVOT} (12)

Here, CSA_{LVOT} is the cross-sectional area of the LVOT (1574). In some variations, blood velocity measurement may be performed in the aortic section of a prosthetic aortic valve by placing the two wireless monitors (1510, 1512) in the aortic section of the valve. In some variations, blood velocity measurement may be performed in the LVOT section of a prosthetic aortic valve by placing the two wireless monitors (1510, 1512) in the LVOT section of the valve. In some variations, the two wireless monitors (1510, 1512) may additionally measure pressure, and the third and fourth wireless monitors (1514, 1516) may not be needed. In some variations, two wireless monitors (1510, 1512), or two transducers of a wireless monitor, may be located about halfway along the length of a prosthetic aortic valve and may interrogate blood velocity using the Doppler technique described herein. In such variations, the two wireless monitors (1510, 1512) may interrogate blood velocity at one or more of in the LVOT, at the aortic valve, in the aorta, and the like.

One or more of these estimated physiological parameters may be used to monitor the operation of a valve, diagnosis of dysfunctions such as obstruction or regurgitation, monitor function of the LV or heart, monitor progression of heart failure, and the like. For example, measurement of SV may be useful to assess if there is high-flow or low-flow. It may, thus, be useful for making a correct diagnosis of a prosthetic valve dysfunction (e.g., aortic stenosis) when used in conjunction with pressure gradient data.

FIG. 16 shows an example of an implantable device (1680) implanted in the LVOT (1674). The implantable device may be in the form of a stent or an expandable implantable device, and may be deployed into the LVOT using a transcatheter delivery mechanism. Such a device may be implanted in a cardiovascular vessel such as one or more of an inflow and/or outflow tract of a ventricle and/or atrium (e.g., LVOT, RVOT), inflow and/or outflow regions of a valve, one or more great vessels (e.g., aorta, pulmonary artery, superior/inferior vena cava), coronary artery, peripheral arteries and/or veins, and the like. Positioning wireless monitors(s) in one or more such locations may be advantageous for monitoring blood flow into and/or out of the heart, estimating cardiac output, measuring blood velocity farther upstream and/or downstream from the wireless monitor location (e.g., using the off-angle Doppler measurement technique described herein), and the like. In some variations, ultrasonic transducers (1602, 1604) may be positioned approximately diametrically opposite to each other on the expandable implantable device, and the Doppler measurement technique described herein may be used to measure blood velocity at one or more locations such as in the aorta (1670), at the aortic valve location, in the LVOT (1674), in the LV, combinations thereof, and the like, by setting the signal roundtrip time. In some variations, other blood velocity measurement techniques (e.g., pulse arrival time measurement) may be used and the transducers may be positioned accordingly, as described before. In some variations, measured blood velocity may also be used to compute one or more of a velocity-time integral, stroke volume, cardiac output, and the like. Transducers (1602, 1604) may belong to the same wireless monitor (not shown) or to separate wireless monitors that may be coupled (e.g., attached) to the expandable implantable device. In some variations, the wireless monitor(s) may alternatively or additionally comprise one or more pressure sensors, which may be configured to measure blood pressure, monitor valve leaflet(s), estimate blood flow, combinations thereof, and the like. Estimation of one or more physiological parameters in one or more locations described herein, may be useful for monitoring one or more of LV and/or RV function, heart failure, valve function, combinations thereof, and the like.

1. A wireless monitoring system, comprising:
a wireless monitor comprising:
a first transducer configured to measure a signal waveform transmitted through one or more of fluid and a physiological structure of a patient; and
a first processor configured to process the measured signal waveform to generate waveform parameter data; and
a wireless device comprising:
a second processor configured to estimate a physiological parameter of the patient based on the waveform parameter data.

2. (canceled)

3. The wireless monitoring system of claim 1, wherein the fluid comprises blood and the physiological structure comprises one or more of a cardiac structure, a vascular structure, and a structure of a cardiovascular implantable device.

4. The wireless monitoring system of claim 1, wherein the waveform parameter data comprises one or more of a Doppler shift, a frequency shift, a phase shift and a time delay, and the physiological parameter comprises a fluid velocity.

5. The wireless monitoring system of claim 4, wherein the wireless monitor comprises a second transducer positioned approximately opposite to the first transducer on or near a vessel wall, wherein the second transducer is configured to transmit a signal waveform and the first transducer is configured to receive a reflected signal waveform, reflected at least in part by fluid flowing through the vessel.

6. The wireless monitoring system of claim 5, wherein the first and second transducers are ultrasonic transducers, and the signal waveform comprises an ultrasonic signal with a carrier frequency of between about 0.1 MHz and about 100 MHz.

7. (canceled)

8. The wireless monitoring system of claim 5, wherein the first and second transducers are configured to perform one or more of one-way pitch-catch measurements, two-way pitch-catch measurements, and pulse-echo measurements for off-angle Doppler estimation of fluid velocity.

9. (canceled)

10. The wireless monitoring system of claim 4, comprising:
a second wireless monitor comprising
a second transducer, wherein the second transducer is configured to transmit a signal waveform and the first transducer is configured to receive a reflected signal waveform, reflected at least in part by fluid flowing through the vessel.

11.-13. (canceled)

14. The wireless monitoring system of claim 1, wherein the signal waveform comprises a set of pulses having a pulse repetition period, the waveform parameter data comprises a set of pulse arrival times of the signal waveform, and the physiological parameter comprises a fluid velocity.

15. The wireless monitoring system of claim 1, wherein the signal waveform comprises a continuous wave signal having a carrier frequency, the waveform parameter data comprises a set of phase shifts of the signal waveform relative to one or more reference phases of the signal waveform, and the physiological parameter comprises a fluid velocity.

16. (canceled)

17. The wireless monitoring system of claim 1, wherein the waveform parameter data comprises one or more transit times of the signal waveform, and the physiological parameter comprises a fluid velocity.

18. The wireless monitoring system of claim 1, wherein the signal waveform is transmitted toward a cardiac structure, and the physiological parameter comprises a cardiac structure parameter.

19. The wireless monitoring system of claim 18, wherein the signal waveform comprises one or more reflected pulses, and the waveform parameter data comprises one or more time durations corresponding to the one or more reflected pulses.

20.-21. (canceled)

22. The wireless monitoring system of claim 18, wherein the cardiac structure comprises a heart chamber and the cardiac structure parameter comprises a volume of the heart chamber.

23. The wireless monitoring system of claim 18, wherein the cardiac structure comprises one or more valve leaflets, and the cardiac structure parameter comprises one or more of valve leaflet motion, thickness, and deterioration.

24. A method of estimating fluid velocity, comprising:
measuring a signal waveform transmitted through patient fluid, measured by a wireless monitor;
processing the measured signal waveform to generate waveform parameter data; and
estimating the fluid velocity of the patient based on the waveform parameter data.

25. The method of claim 24, wherein the waveform parameter data comprises one or more of a Doppler shift, a frequency shift, a phase shift and a time delay.

26. (canceled)

27. The method of claim 24, wherein the signal waveform comprises a set of pulses having a pulse repetition period, and the waveform parameter data comprises a set of pulse arrival times of the signal waveform.

28. The method of claim 24, wherein the signal waveform comprises a continuous wave signal having a carrier frequency, and the waveform parameter data comprises a set of phase shifts of the signal waveform relative to one or more reference phases of the signal waveform.

29.-30. (canceled)

31. A method of estimating a cardiac structure parameter, comprising:
measuring a signal waveform transmitted toward a cardiac structure of a patient using a wireless monitor;
processing the measured signal waveform to generate waveform parameter data; and estimating the cardiac structure parameter based on the waveform parameter data.

32.-44. (canceled)

45. The wireless monitoring system of claim 1, wherein the wireless device is configured to be disposed external and physically separate from the wireless monitor and within or on one or more of a cardiac structure and a vascular structure.

46-58. (canceled)
